(19) United States
(12) Patent Application Publication
(10) Pub. No.: US 2024/0101664 A1
(43) Pub. Date: Mar. 28, 2024

(54) TREATMENT OF PERITONEAL, PLEURAL, AND PERICARDIUM ADHESIONS

(71) Applicant: UTI LIMITED PARTNERSHIP, Calgary (CA)

(72) Inventors: Joel S. ZINDEL, Koniz (CH); Paul KUBES, Cochrane (CA)

(21) Appl. No.: 18/257,107

(22) PCT Filed: Dec. 14, 2021
(86) PCT No.: PCT/CA2021/051805

§ 371 Date: Jun. 13, 2023

Related U.S. Application Data
(60) Provisional application No. 63/125,020, filed on Dec. 14, 2020.

(51) Int. Cl. C07K 16/28; A61P 41/00

(52) U.S. Cl. CPC C07K 16/28; A61P 41/00; A61K 2039/505; C07K 2317/76

(57) ABSTRACT

The present disclosure relates generally to treatment of peritoneal, pleural, and pericardium adhesions.

### CROSS REFERENCE TO RELATED APPLICATION

This application claim priority to United States Provisional patent application U.S. 63/125,020, filed Dec. 14, 2020, the entire contents of which is hereby incorporated by reference.

### FIELD

The present disclosure relates generally to treatment of peritoneal, pleural, and pericardium adhesions.

### BACKGROUND

Surgeries within body cavities such as the abdominal cavity are often life saving procedures. In some patients, however, abdominal surgery can lead to a fibrotic complication called post-surgical peritoneal adhesions. These are intra-abdominal scar bands that form when surgical injury disturbs the mesothelial lining of organs and the abdominal wall.¹ Adhesions impair free movement of organs and cause a major health burden for patients and health care providers². They are the leading cause of potentially life-threatening intestinal occlusions^{1,3,4} requiring emergency surgery for treatment. In the United States for example, post-surgical adhesions are responsible for over 300,000 additional abdominal operations per year with direct annual costs of several billion dollars². In addition, adhesions frequently lead to chronic post-operative abdominal pain. The mechanism of adhesion formation is poorly understood and up to date no effective treatment exists. The dogma over the last two decades has been that surgery causes activation of the coagulation cascade which leads to the deposition of a fibrin clot. This fibrin clot then serves as a scaffold for a fibrotic reaction which results in aberrant scar tissue. However, several drugs targeting the formation of fibrin clots have failed to prevent peritoneal adhesions in clinical trials.

The peritoneal cavity fluid is under constant motion with shear stress levels comparable to arteries rather than veins⁵.

### SUMMARY

In one aspect there is provided a method of treating a subject having or suspected of having a disease or disorder associated with peritoneal adhesions, comprising: administering an inhibitor of a scavenger receptor comprising a SRCR domain recognizing negatively charged macromolecules.

In one aspect there is provided a method of treating a subject having or suspected of having a disease or disorder associated with pericardium adhesions, comprising: administering an inhibitor of a scavenger receptor comprising a SRCR domain recognizing negatively charged macromolecules.

In one aspect there is provided a method of treating a subject having or suspected of having a disease or disorder associated with pleural cavity adhesions, comprising: administering an inhibitor of a scavenger receptor comprising a SRCR domain recognizing negatively charged macromolecules.

In one aspect there is provided a method of treating a subject having or suspected of having peritoneal adhesions, comprising: administering an inhibitor of a scavenger receptor comprising a SRCR domain.

In one aspect there is provided a method of treating a subject having or suspected of having pericardium adhesions, comprising: administering an inhibitor of a scavenger receptor comprising a SRCR domain.

In one aspect there is provided a method of treating a subject having or suspected of having plural cavity adhesions, comprising: administering an inhibitor of a scavenger receptor comprising a SRCR domain.

In one example, wherein the disease or disorder associated with peritoneal adhesions comprises adhesions that results from abdomino-pelvic surgery, radiation therapy, and/or inflammatory processes.

In one example, wherein the disease or disorder associated with pericardium adhesions comprises adhesions that results from abdomino-pelvic surgery, radiation therapy, and/or inflammatory processes.

In one example, wherein the disease or disorder associated with pleural cavity adhesions comprises adhesions that results from abdomino-pelvic surgery, radiation therapy, and/or inflammatory processes.

In one example, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

In one example, wherein said inhibitor comprises or consists of Polyl.

In one example, where said inhibitor comprises or consists of an antibody.

In one example, wherein said antibody is a polyclonal antibody or a monoclonal antibody.

In one example, wherein the subject is a human.

In one aspect there is provided a use of an inhibitor of a scavenger receptor comprising a SRCR domain recognizing negatively charged macromolecules, for treating a subject having or suspected of having a disease or disorder associated with peritoneal adhesions.

In one aspect there is provided a use of an inhibitor of a scavenger receptor comprising a SRCR domain recognizing negatively charged macromolecules, in the manufacture of a medicament for treating a subject having or suspected of having a disease or disorder associated with peritoneal adhesions.

In one aspect there is provided a use of an inhibitor of a scavenger receptor comprising a SRCR domain recognizing negatively charged macromolecules for treating a subject having or suspected of having a disease or disorder associated with pericardium adhesions.

In one aspect there is provided a use of an inhibitor of a scavenger receptor comprising a SRCR domain recognizing negatively charged macromolecules in the manufacture of a medicament for treating a subject having or suspected of having a disease or disorder associated with pericardium adhesions.

In one aspect there is provided a use of an inhibitor of a scavenger receptor comprising a SRCR domain recognizing negatively charged macromolecules treating a subject having or suspected of having a disease or disorder associated with pleural cavity adhesions.

In one aspect there is provided a use of an inhibitor of a scavenger receptor comprising a SRCR domain recognizing negatively charged macromolecules in the manufacture of a medicament for treating a subject having or suspected of having a disease or disorder associated with pleural cavity adhesions.

In one aspect there is provided a use of an inhibitor of a scavenger receptor comprising a SRCR domain for treating a subject having or suspected of having peritoneal adhesions.

In one aspect there is provided a use of an inhibitor of a scavenger receptor comprising a SRCR domain in the manufacture of a medicament for treating a subject having or suspected of having peritoneal adhesions.

In one aspect there is provided a use of an inhibitor of a scavenger receptor comprising a SRCR domain for treating a subject having or suspected of having pericardium adhesions.

In one aspect there is provided a use of an inhibitor of a scavenger receptor comprising a SRCR domain in the manufacture of a medicament for treating a subject having or suspected of having pericardium adhesions.

In one aspect there is provided a use of a scavenger receptor comprising a SRCR domain for treating a subject having or suspected of having plural cavity adhesions.

In one example, wherein the disease or disorder associated with peritoneal adhesions comprises adhesions that results from abdomino-pelvic surgery, radiation therapy, and/or inflammatory processes.

In one example, wherein the disease or disorder associated with pericardium adhesions comprises adhesions that results from abdomino-pelvic surgery, radiation therapy, and/or inflammatory processes.

In one example, wherein the disease or disorder associated with pleural cavity adhesions comprises adhesions that results from abdomino-pelvic surgery, radiation therapy, and/or inflammatory processes.

In one example, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

In one example, wherein said inhibitor comprises or consists of Polyl.

In one example, where said inhibitor comprises or consists of an antibody.

In one example, wherein said antibody is a polyclonal antibody or a monoclonal antibody.

In one example, wherein the subject is a human.

In one aspect there is provided a kit for treating a subject having or suspected of having a disease or disorder associated with peritoneal adhesions, comprising: an inhibitor of a scavenger receptor comprising a SRCR domain recognizing negatively charged macromolecules, a container, and optionally instructions for the use thereof.

In one aspect there is provided a kit for treating a subject having or suspected of having a disease or disorder associated with pericardium adhesions, comprising: an inhibitor of a scavenger receptor comprising a SRCR domain recognizing negatively charged macromolecules, a container, and optionally instructions for the use thereof.

In one aspect there is provided a kit for treating a subject having or suspected of having a disease or disorder associated with pleural cavity adhesions, comprising: an inhibitor of a scavenger receptor comprising a SRCR domain recognizing negatively charged macromolecules, a container, and optionally instructions for the use thereof.

In one aspect there is provided a kit for treating a subject having or suspected of having peritoneal adhesions, comprising: an inhibitor of a scavenger receptor comprising a SRCR domain, a container, and optionally instructions for the use thereof.

In one aspect there is provided a kit for treating a subject having or suspected of having pericardium adhesions, comprising: an inhibitor of a scavenger receptor comprising a SRCR domain, a container, and optionally instructions for the use thereof.

In one aspect there is provided a kit for treating a subject having or suspected of having plural cavity adhesions, comprising: an inhibitor of a scavenger receptor comprising a SRCR domain, a container, and optionally instructions for the use thereof.

In one example, wherein the disease or disorder associated with peritoneal adhesions comprises adhesions that results from abdomino-pelvic surgery, radiation therapy, and/or inflammatory processes.

In one example, wherein the disease or disorder associated with pericardium adhesions comprises adhesions that results from abdomino-pelvic surgery, radiation therapy, and/or inflammatory processes.

In one example, wherein the disease or disorder associated with pleural cavity adhesions comprises adhesions that results from abdomino-pelvic surgery, radiation therapy, and/or inflammatory processes.

In one example, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

In one example, wherein said inhibitor comprises or consists of Polyl.

In one example, where said inhibitor comprises or consists of an antibody.

In one example, wherein said antibody is a polyclonal antibody or a monoclonal antibody.

In one example, wherein the subject is a human.

### BRIEF DESCRIPTION OF THE DRAWINGS

Embodiments of the present disclosure will now be described, by way of example only, with reference to the attached Figures.

FIG. 1. Gata6⁺ cavity macrophages rely on passive transport for recruitment to laser-induced focal injury. (A) Anatomy of the peritoneal cavity. (B) Illustration of abdominal pouch intravital microscopy (IVM) model. (C) Microscopic situs shows stable abdominal wall (asterisk) and moving cavity macrophages (arrow). (D) Open-pouch IVM model with fluid movement, transmesothelial laser injury (dashed circle), cavity macrophages stained with anti-F4/80 and dead cells with Sytox green. Scale bar: 50 μm. (E) Quantification of (D). (F) Closed-pouch IVM model with no fluid movement, transmesothelial laser injury (dashed circle), Staining same as (D). Scale bar: 50 μm. (G) Quantification of (F). (H) Abdominal-pouch IVM preparation, laser injury of the rectus abdominus muscle in the abdominal wall (dashed circle) in Ly6G^{tdTomato} fluorescent neutrophil reporter. (I) Quantification of (H). (J) Resonant scanning mode with high frame rate (50 frames per second). Representative imaging (upper panel) and tracking (lower panel) of one floating peritoneal macrophage (arrow) in relation to cells already aggregated (dashed circle). Scale bar: 50 μm. (K) Peritoneal cavity macrophages in different stages of aggregation. Scale bar: 10 μm. (L) 3D-reconstruction of transmesothelial abdominal wall injury (upper panel) and the resulting macrophage aggregation after 30 min (lower panel). Scale bar: 50 μm. (M) scatter plot and fitted linear regression line of cell count at injury versus injury size. Data are mean±sd. n=5, 4, and 3 in (E), (G), and (I) respectively. P-values by repeated measure one-way ANOVA (versus first time point); **P<0.01, ***P<0.001, n.s. P≥0.05. Reported F-values are for over time variability. P-values and R² in (M) by linear regression model. Symbols in (M) represent individual observations (n=89) representing a total of n=29 control (wild-type, untreated) animals of different experiments. All data are representative of N≥3 independent experiments. Fps: frames per second. SHG: second harmonic generation (collagen).

FIG. 2. Cavity macrophages as extravascular platelets. (A) Intravital microscopy (IVM) of epigastric blood vessel. Transendothelial laser injury (dashed line). Platelets are stained with intravenous injection of anti-CD49b antibody. Scale bar: 50 μm. (B) Open-pouch IVM. Transmesothelial laser injury (dashed line). Macrophages were stained with intraperitoneal injection of anti-F4/80 antibody. Scale bar: 50 μm. (C) Quantification of aggregate/clot volume in (A) and (B) overtime. (D) 3D-reconstruction of open pouch IVM model images acquired at 30 min after laser injury. Injury size was increased to span transmesothelial and transendothelial compartment at the same time. Macrophages were stained by intraperitoneal anti-F4/80 injection. Platelets stained by CD41YFP fluorescent reporter. Scale bar: 50 μm. (E) Platelet and macrophage aggregation traces (recording time=6 min). Aggregometer was equilibrated with isolated platelets or peritoneal macrophages. After 1 minute, agonists were added. Representative of two independent experiments are depicted. (F) Macroscopic image of cuvettes at 10 min. (G and H), phase contrast microscopy of cuvette content at 10 min. Scale bar: 50 μm. Images and curves are single mice, representative of N≥2 independent experiments. SHG: second-harmonic generation (collagen). Auto: autofluorescence.

FIG. 3. Aggregation of cavity macrophage does not rely on known mammalian cell-adhesion molecules but relies on evolutionary conserved scavenger receptors. (A) Normalized macrophage count (cell count divided by injury size) at 30 min post injury. ANOVA: P<0.001, F-value=24.3. (B and C) RNA-Seq gene expression levels (n=4 biological replicates) of class A and class B scavenger receptors, respectively. (D) Open pouch intravital microscopy. Images acquired at 30 min after trans mesothelial laser injury. Animals treated with either poly(C) or poly(I). Scale bar: 50 μm. (E) Normalized macrophage count at 30 min post injury after poly(C) and poly(I) treatment (D). t test: t=6.26, P=0.002 (F) Normalized macrophage count at 30 min post injury. Blocking antibodies were administered at 20 min before injury. ANOVA: P<0.001. F-value=11.0. (G) Illustration of peritoneal focal thermal injury model. (H) peritoneal flap intravital microscopy at 3 days after peritoneal focal thermal injury. Animals treated with either poly(C) or poly(I). Images are representative of quantification shown in (I) and (J). Scale bar: 500 μm. (I and J) Quantification of wound area (I) and mesothelial coverage (J) at 3 days after injury. Data are mean±individual values (mice). t test of (1): P=0.028, t=−2.63. t test of (J): P=0.00098, t=4.75. P-values by unpaired t tests or one-way ANOVA with Tukey's post hoc test; *P<0.05, **P<0.01, ***P<0.001, n.s. P≥0.05. Data are representative N=2 independent experiments. RPKM: Reads per kilobase of transcript per million mapped reads. SHG: second-harmonic generation (collagen).

FIG. 4. Super aggregates and scar formation. (A) Illustration of the abdominal window intra vital microscopy (AW-IVM). (B) Size comparison open-pouch (simple) macrophage aggregate versus AW-IVM (super) macrophage aggregate. Scale bar: 50 μm. (C) Quantification of representative images shown in (B). Wilcoxon test: P=0.004. (D) AW-IVM at 1 h after window implantation. Macrophages are stained with intraperitoneal injection of anti-F4/80 antibody. Overtime, macrophages attached to the window (arrow) merge with macrophages attached an intra-abdominal organ (asterisk) to form super-aggregates/adhesion precursor (dashed lines). Scale bar: 100 μm. (E) AW-IVM at 3 days after window implantation. Macrophages and mesothelium are stained with intraperitoneal injection of anti-F4/80 and anti-podoplanin antibody, respectively. Dashed lines indicate adhesions. Scale bar: 100 μm. (F) AW-IVM at 7 days after window implantation. (G) Illustration of (D) to (F). Second-harmonic generation (SHG) visualizes de novo collagen deposition. Scale bar: 500 μm. Data are median±individual values (mice). P-values by Wilcoxon test. **P<0.01. Data are representative of N=2 independent experiments.

FIG. 5. Clinical implications of macrophage aggregation inhibition. (A) Illustration of peritoneal-button injury model. (B) Macroscopic and microscopic images of peritoneal adhesion (dashed lines) at 7 days after peritoneal button injury. Adhesion ranges from peritoneal button (arrow) to omentum (arrowhead). A yellow pipette tip (orange lines) was inserted below the adhesion. Microscopic image shows section stained with Masson's trichrome. Scale bars: 1 cm (macroscopic) and 300 μm (microscopic). (C) Cleared whole-mount microscopy of peritoneal buttons, harvested at 7 days after surgery. Scale bar: 200 μm. (D) Representative flow cytometry scatter plots, pre-gated on size, singlets, live, CD45⁺CD3⁻CD19⁻. (E) Quantification of peritoneal macrophages (F4/80^{hi}CD102^{hi}) as representatively shown in (D). ANOVA: P=0.013, F-value=5.48. (F) Whole-mount microscopy of peritoneal button and peritoneal adhesion at 7 days after surgery. Blue lines indicate position of sutures. Scale bar: 500 μm. (G) Quantification of peritoneal adhesions 7 days after surgery. ANOVA: P<0.001. PBS: PBS liposomes. CLD: clodronate liposomes. Cre⁺: Csf1R^{Cre+}iDTR, Cre⁻: Csf1R^{Cre-}iDTR: inducible diphtheria toxin receptor. Data are mean±individual values (mice). P-values by ANOVA with Tukey's post hoc test; *P<0.05, **P<0.01, n.s. P≥0.05. Data are representative of N=2 independent experiments.

FIG. 6 Cavity macrophages in suspension rely on passive transport by convective fluid movement. (A) Illustration of the abdominal imaging window intra vital microscopy (AIW-IVM). (B) AW-IVM, Macrophages are stained by Gata6^{Venus} reporter. At t=0, 3×10⁶ cavity macrophages were adoptively transferred (i.p. injection) from another Gata6^{Venus} mouse. Dashed circles indicate appearance of transplanted macrophages. (C) Illustration of mouse abdominal wall. (D) Whole-mount imaging, cross-sectional planes as indicated in (C). (E) Open pouch IVM. High-fps imaging and automated tracking of single cells. Individual tracks are displayed as relative displacement (Δ√x²+y²) over time (Δt). (F) Open pouch IVM. Nuclear staining delineates mesothelium which is targeted with constant illumination with a 16× digital zoom, high laser power (77 mW) at 1045 nm for 5 s. Resulting in a trans mesothelial breach/injury of the abdominal wall. (G) Open pouch IVM with laser injury, Gata6^{Venus} bone marrow transplanted C57BL/6 mice at 8 weeks prior to imaging. No intraperitoneal injection/manipulation. (H) Open pouch IVM with laser injury, Gata6^{Venus} bone marrow transplanted C57BL/6 mice received either anti-F4/80 or saline intraperitoneally, number of Venus⁺ nuclei in lesion at 30 min were counted. (I) Closed pouch IVM. High-fps imaging and tracking of single cells. Individual tracks are displayed as relative displacement (Δ√x²+y²) overtime (Δt). (J) Closed pouch IVM. Cavity macrophages (F4/80^{hi}) were tracked and relative distance to laser injury over time is displayed. (K) IVM with injury to the abdominal wall (instead of trans mesothelial). Neutrophils (Ly6 g^{tdTomato+}) were tracked and relative distance to laser injury over time is displayed. Data are mean±individual values (mice). P-values by unpaired t tests. n.s. P≥0.05. Data are representative of 2 independent experiments. SHG: second harmonic generation (collagen). Fps: frames per second. RAM: rectus abdominis muscle. Int: intestine. PC: peritoneal cavity.

FIG. 7 Free-floating versus crawling peritoneal macrophages. (A) Open-pouch intravital microscopy (IVM) in resonant scanning mode with ultra-high frame rate (55 frames per second) allows to track free-floating peritoneal macrophages. Scale bar: 50 μm. Image representative of n=4 biological replicates of N=2 independent experiments. (B) Urea nitrogen concentration was measured simultaneously in the blood and peritoneal lavage with 1 ml PBS. Data represent n=40 biological replicates of N=2 independent experiments. R-squared by linear regression, P<0.0001. (C) Closed-pouch IVM at 4, 20 and 27 min after injury (dashed circle) with tracks (right panel) over the whole time-period. (D) Relative displacement of cells in (C) overtime. (C) and (D) represent n=1 representative biological replicate of N=1 independent experiment.

FIG. 8. Cavity macrophages function as extravascular platelets. (A) 3D-reconstruction of intravital microscopy (IVM) of epigastric blood vessel. Trans endothelial laser injury. Platelets are stained with intravenous injection of anti-CD49b antibody. Scale bar: 50 μm. (B) Open pouch IVM. 3D-reconstruction of open pouch IVM model with trans mesothelial injury. Macrophages were stained with intraperitoneal injection of anti-F4/80 antibody. Scale bar: 50 μm. (C) Raw light transmittance of curves shown as percent maximal aggregation in FIG. 2E. Platelet and macrophage aggregation traces (recording time=6 min). Aggregometer was equilibrated with isolated platelets or peritoneal macrophages. After 1 minute, agonists were added. Representative of n=5 biological replicates of N=2 independent experiments are depicted. (D) Microscopic analysis of cuvette content after incubating 10 minutes in aggregometer, macrophages stained with F4/80. Scale bar: 50 μm. (E) Quantification of microscopy images of cuvette content. Percentage of aggregated vs free macrophage (F4/80⁺) count per field of view. (F) Aggregometry traces of neutrophils isolated from (murine) blood. Representative images of n=4 biological replicates of N=2 independent experiments are shown. (G) Transmission electron microscopy of cuvette content after aggregometry. Scale bar overview: 5 μm. Scale bar insets: 2 μm. Data are mean±individual values (mice). P-values by unpaired t tests. ****P<0.0001. SHG: second harmonic generation (collagen). (H) shows that depletion of platelets did not affect the formation of the macrophage aggregate.

FIG. 9. Aggregation of cavity macrophage does not rely on known mammalian cell-adhesion molecules. (A) RNA-Seq gene expression levels (sorted cavity macrophages) of known mammalian integrins. (B) Normalized macrophage count (cell count divided by injury size) at 30 min post injury. Mice received either anti-CD18+anti-CD29 monoclonal antibody or the same amount of the respective isotypes. t test: P=0.21, t=1.46. (C) Expression levels of selectins. (D) Normalized cell count at 30 min post injury. Mice either received anti-CD162 monoclonal antibody or isotype control. t test: P=0.86, t=0.19. (E) Expression levels of immunoglobulin (Ig)-like adhesion molecules. (F to I) Normalized cell count at 30 min post injury. Mice were either specific gene knockouts or neutralized by specific monoclonal antibodies. Data were compared to the C57BL/6 wild type strain or the respective isotype control. ANOVA of (F): df=2, F=1.7. t test of (G): P=0.49, t=−0.77. t test of (H): P=0.60, t=0.59. ANOVA of (I): P=0.48, P=0.25, df=2, F=0.83 (J) Open pouch intravital microscopy. Images acquired at 30 min after trans mesothelial laser injury. Animals treated with either saline or 400 U Heparin. Data are mean±individual values (mice). P-values by unpaired t tests or one-way ANOVA with Tukey's post hoc test; n.s. p≥0.05. Data from N=2 independent experiments, except for (A), (C), and (E) which represent N=1 independent experiment with n=3-4 animals per group. RPKM: Reads per kilobase of transcript per million mapped reads. SHG: second harmonic generation (collagen). α: neutralizing monoclonal antibody.

FIG. 10. Inhibition, expression, and regulation of scavenger receptors. (A) Phase-contrast microscopy images of cuvette content after aggregometry (37° C., 400 rpm, 10 min). Scale bar 500 μm. (B) Quantification of (A). P-values by unpaired t test: P=0.0019, t=5.28. (C) Multi-dimensional scaling plot of transcriptomic profiles of different tissue macrophage populations and monocytes. (D) Normalized expression levels of Marco and Msr1 for populations shown in (C). P-values by binomial linear regression model. (E and F) Protein expression levels of Marco (E) and Msr1 (F) both before and after stimulation with 100 μM ATP. Signal intensity was measured on the surface and in the cytosol on CD45⁺Ly6C/G⁻CD11b⁺F480^{hi}CD102⁺ cells by flow cytometry (n=4 per group). P-values by paired t tests. Data are mean±individual values (mice). *P<005, **P<0.01, ***P<0.001, n.s. P>0.05. (A) to (D) are representative of N=1, (E) and (F) of N=2 independent experiments. ATP: adenosine triphosphate. CPM: counts per million.

FIG. 11. Healing of focal thermal hepatic injury. (A) Illustration of focal hepatic thermal injury. (B) Liver intravital microscopy at 7 d after injury. Blood vessels stained with anti-CD31 antibody. Collagen with second harmonic generation (SHG). Scale bar: 500 μm (C and D) Quantification of wound area defined as blood vessel free area (C) and quantification of wound area already covered with collagen (D). t test of (C): P=0.001, i=−4.66. t test of (D): P=0.002, t=4.08. Data are mean±individual values (mice). P-values by unpaired t tests. **P<0.01. Data are representative of N=2 independent experiments (n=7 per group). SHG: second-harmonic generation (collagen).

FIG. 12. Macrophage super-aggregation precedes collagen deposition. (A) Abdominal imaging window intravital microscopy (AIW-IVM). View on intestinal surface. Macrophages stained with anti-F4/80 antibody. Dashed lines indicate a serosal blood vessel on intestinal surface. Scale bar: 100 μm (B) AIW-IVM. Adoptive transfer of 3×10⁶ cavity macrophages, stained ex vivo with anti-F4/80 antibody. Insets show change overtime. Scale bar: 50 μm. (C) AIW-IVM 2 hours after window implantation and adoptive transfer of Gata6^{Venus} peritoneal macrophages into C57/BL6 wild-type mouse. All macrophages (transplanted Venus⁺ and resident Venus-) were stained with anti-F4/80 antibody intraperitoneally. Scale bar: 70 μm. (D) Whole-mount images of peritoneal buttons one, three, five, and seven days post-surgery. Dashed lines highlight areas of adhesion formation. Scale bar: 500 μm. Images are representative of n≥3 biological replicates. (E) Magnification and illustration of last panel in (D). Scale bar: 500 μm. (A) to (C) represent N=2 independent experiments, and (D) and (E) represent N=1 independent experiment.

FIG. 13. Adhesion formation associated with cavity macrophage aggregation. (A) Flow cytometry plots of peritoneal lavage at 3 h after peritoneal button injury. Mice were preoperatively treated with either Poly-(C) or Poly-(I). Cells were pre-gated on size, live, CD45⁺CD3⁻CD19⁻Ly6G⁻. (B) Quantification of LPM (CD102^{hi}F4/80^{hi}) cells in peritoneal fluid 3 hours post-surgery as representatively shown in (A). (C) Representative macroscopic image of peritoneal button at 7 days post-surgery. Mice were preoperatively treated with either Poly-(C) or Poly-(I). (D and E) Peritoneal lavage flow cytometry results at 24 hours after administration of diphtheria toxin in Csf1R^{HBEGF/mCherry} iDTR mice. Cells were pre-gated on size, live, CD45⁺CD19⁻Ly6G⁻. (F) Whole-mount microscopy of peritoneal buttons three and twenty-four hours after surgery. (G) Quantification of peritoneal adhesions 7 days after surgery (n=4 per group). t test: P=1.0, t=0. Data represent mean+individual values. (B) to (C) represent N=2 independent experiments, and (D) to (G) represent N=1 independent experiment. LPM: large peritoneal macrophages.

FIG. 14 depicts the effect of Poly(I) on human peritoneal fluid sample.

### DETAILED DESCRIPTION

Herein we describe that resident macrophages are extravascular counterparts to platelets. We show how peritoneal macrophages rapidly form thrombus-like aggregates in response to injury. The dynamics of these aggregates mirror and rival the speed of platelet thrombus formation in the adjacent vasculature. We show that these macrophage aggregates are an initiating event in post-surgical adhesion formation and that adhesions can be prevented by either depleting peritoneal macrophages or by inhibiting their aggregation.

In some examples, there is provided in treating a subject having or suspected of having a disease or disorder associated with peritoneal adhesions.

In one example, a subject is treated with an inhibitor of a scavenger receptors. In a specific example, the inhibitor of a scavenger receptor comprises or consists of Polyl (Polyinosinic acid). In another example the inhibitor of a scavenger receptor comprises or consists of an antibody, such as a polyclonal antibody or monoclonal antibody.

In one example, the disease or disorder associated with peritoneal adhesions comprises adhesions that results from abdomino-pelvic surgery, radiation therapy, and/or inflammatory processes.

In one example, there is provided a method of treating a subject having or suspected of having a disease or disorder associated with pericardium adhesions, comprising: administering an inhibitor of a scavenger receptor comprising a SRCR domain recognizing negatively charged macromolecules.

In one example there is provided a method of treating a subject having or suspected of having a disease or disorder associated with pleural cavity adhesions, comprising: administering an inhibitor of a scavenger receptor comprising a SRCR domain recognizing negatively charged macromolecules.

In one example there is provided a method of treating a subject having or suspected of having peritoneal adhesions, comprising: administering an inhibitor of a scavenger receptor comprising a SRCR domain.

In one example there is provided a method of treating a subject having or suspected of having pericardium adhesions, comprising: administering an inhibitor of a scavenger receptor comprising a SRCR domain.

In one example there is provided a method of treating a subject having or suspected of having plural cavity adhesions, comprising: administering an inhibitor of a scavenger receptor comprising a SRCR domain.

An adhesion or adhesions refer to a state where surfaces of tissues that should be separated from each other are connected or fused to each other via fibrous tissue.

In some examples, adhesions are caused by an injury generated on a surface of a tissue upon a surgical operation, inflammation caused by an injury, and/or inflammation caused by drying of a tissue surface upon a surgical operation.

In some examples, adhesions are formed in association with such injury or inflammation upon which an exudate containing fibrin is emitted on a surface of a tissue, where this exudate is organized such that the tissue surfaces are connected or fused.

In some aspects, there is described a method of preventing or reducing adhesion. Adhesion prevention does not necessarily require complete prevention of adhesion formation, and may apply as long as formation of adhesion is prevented as compared to a state where an anti-adhesion material and/or method as described herein is not applied.

In some examples, prevention of adhesion refers to amelioration of adhesions, which may mean, for example, amelioration of at least one selected from frequency, area and degree of the adhesions.

In some examples prevention of adhesion may be, for example, lowering of the average adhesion grade as compared to the average adhesion grade without application of an anti-adhesion material and/or method as described herein.

In some examples, prevention of adhesion may be, for example, lowering of the average adhesion extent as compared to the average adhesion extent without application of the anti-adhesion material of the present invention when adhesion extent is evaluated as described in the example.

In some examples, prevention of adhesion may refer to prevention of adhesion resulting from a surgical operation. In another example, prevention of adhesion refers to prevention of peritoneal adhesion, for example resulting from a surgical operation.

In another example, prevention of adhesion refers to prevention of an adhesion following a surgery.

In another example, adhesions targeted include an adhesion of a resected site of an organ targeted by a surgery and a de novo adhesion (adhesions formed at variety of sites other than the surgical site, in the periphery, the body cavity such as the abdominal cavity and the body).

In one example, a “tissue involved in a surgical operation” refers to a tissue that has a wound on its surface due to the surgical operation, or a tissue that has inflammation or that has a risk of inflammation due to drying of the surface due to a surgical operation. A tissue involved in a surgical operation may be an organ wrapped in peritoneum (for example, stomach, jejunum, ileum, appendix, colon, liver, gallbladder, spleen, duodenum, uterus, fallopian tube, ovary, abdominal wall and pancreas), an organ covered with a pleura (lung, chest wall), an organ covered with pericardium (heart, pericardium sac) or the like.

In some examples, the term “surgical site” or “abdominal surgical site” refers to tissues around a surgical incision, organs adjacent to the incision, and generally surfaces in the peritoneal cavity.

In some examples, the term “peritoneal adhesions” refer to a condition in which pathological bonds form between the omentum, the small and large bowels, the abdominal wall, and other intra-abdominal organs.

In some examples, the term “post-surgical adhesions” refers to adhesions that develop following surgery. In particular, abdominal adhesions are bands of fibrous scar tissue that form on organs in the abdomen. They can cause organs to stick to one another or to the wall of the abdomen.

In some examples, reducing peritoneal adhesions refers to the reduction of post-surgical adhesions by about 10% to about 99%, for example, about 10%, 20%, 25%, 30%, 35%, 40%, 45%, 50%, 55%, 60%, 65%, 75%, 80%, 85%, 90%, 95%, 96%, 97%, 98%, or 99% through use of the compounds, compositions and/or methods as described herein.

In some examples, inhibition of post-surgical adhesions by the compounds, compositions and/or methods as described herein refers to the reduction of post-surgical adhesions by about 10% to above 99%, for example about 10%, 20%, 25%, 30%, 35%, 40%, 45%, 50%, 55%, 60%, 65%, 75%, 80%, 85%, 90%, 95%, 96%, 97%, 98%, or 99%.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “treatment”, “treat”, or “treating” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

In one example, a compound or composition as described herein is given at the time of surgery.

The term “amelioration” or “ameliorates” as used herein refers to a decrease, reduction or elimination of a condition, disease, disorder, or phenotype, including an abnormality or symptom.

The term “symptom” of a disease or disorder is any morbid phenomenon or departure from the normal in structure, function, or sensation, experienced by a subject and indicative of disease.

A “Treatment regimen” as used herein refers to a combination of dosage, frequency of administration, or duration of treatment, with or without addition of a second medication.

Non-limiting symptoms that may be associated with adhesions, such as peritoneal adhesions includes acute small bowel obstruction leading acute emesis, pain and potentially life-threatening gangrene. Subacute obstruction can lead to chronic (persistent or intermittent) bloating, abdominal cramping or borborygmi, altered bowel habits including constipation or frequent stools, nausea with our without early satiety, bowel obstructions which may be transient, partial, or complete, female infertility and dyspareunia, rectal bleeding and dyschezia during menses.

A compound or composition may be administered alone or in combination with other treatments, either simultaneously or sequentially, dependent upon the condition to be treated.

In treating a subject, a therapeutically effective amount may be administered to the subject.

In one example, a subject is treated with inhibiting SRCR receptors. And then including other claims for the compound Polyl and perhaps monoclonal antibodies that could inhibit the scavenger receptors

As used herein, the term “therapeutically effective amount” refers to an amount that is effective for preventing, ameliorating, or treating a disease or disorder (e.g., inflammatory bowel disease, e.g., ulcerative colitis or Crohn's disease, e.g., cancer).

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

Compounds and/or compositions comprising compounds disclosed herein may be used in the methods described herein in combination with standard treatment regimes, as would be known to the skilled worker.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

Abstract

Most multicellular organisms have a major body cavity that harbors immune cells. In primordial species like purple sea urchins, these cells perform phagocytic functions but are also crucial in repairing injuries. In mammals, the peritoneal cavity contains large numbers of resident GATA6⁺ macrophages, which may play a similar role. However, it is unclear how cavity macrophages suspended in the fluid phase (peritoneal fluid) identify and migrate towards injuries. Here, we show using intravital microscopy, that cavity macrophages in fluid rapidly form thrombus-like structures in response to injury using primordial scavenger receptor (SRCR) domains. Aggregates of cavity macrophages physically sealed injuries and promoted rapid repair of focal lesions. In iatrogenic surgical situations, these cavity macrophages formed extensive aggregates that promoted the growth of intra-abdominal scar tissue termed peritoneal adhesions

Here, we show that cavity macrophages are extravascular counterparts to platelets, forming rapid thrombus-like structures in response to injury that mirror and rival the speed of platelet thrombus formation in the adjacent vasculature. We show that cavity macrophage aggregation depends on primordial scavenger receptor domains, with many homologues expressed by sea urchin coelomocytes. Aggregates of cavity macrophages physically seal injuries and promote repair of focal lesions. However, with more complex injuries, cavity macrophages form extensive aggregates that promote the growth of intra-abdominal scar tissue called adhesions which results in significant morbidity.

Imaging of Gata6⁺ Cavity Macrophages

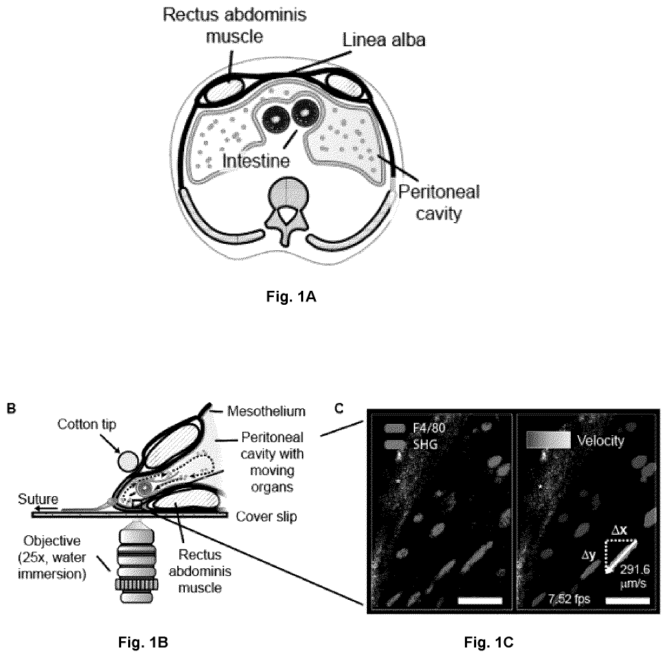

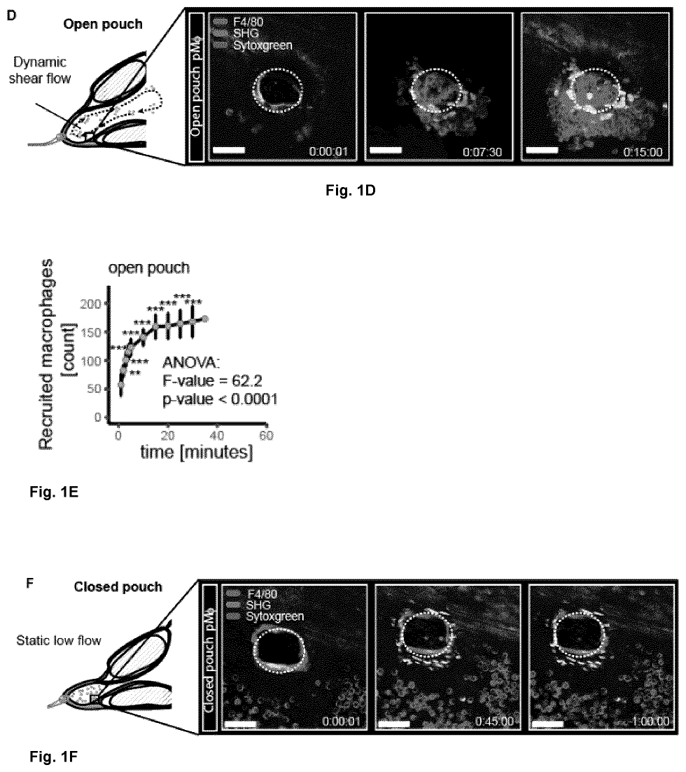

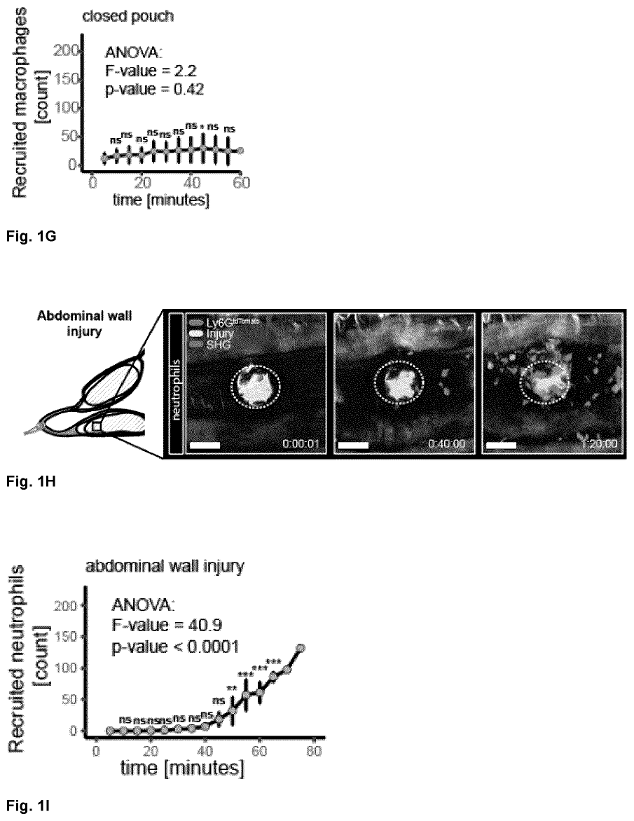

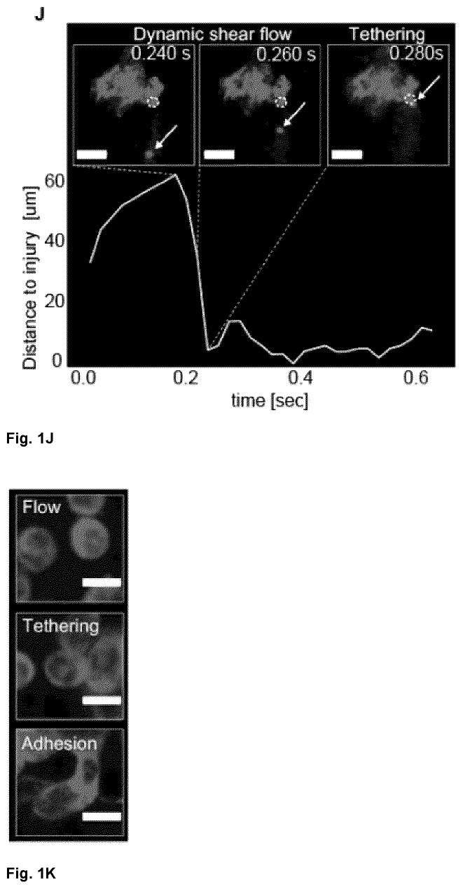

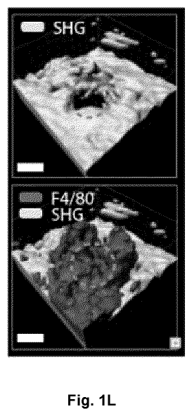

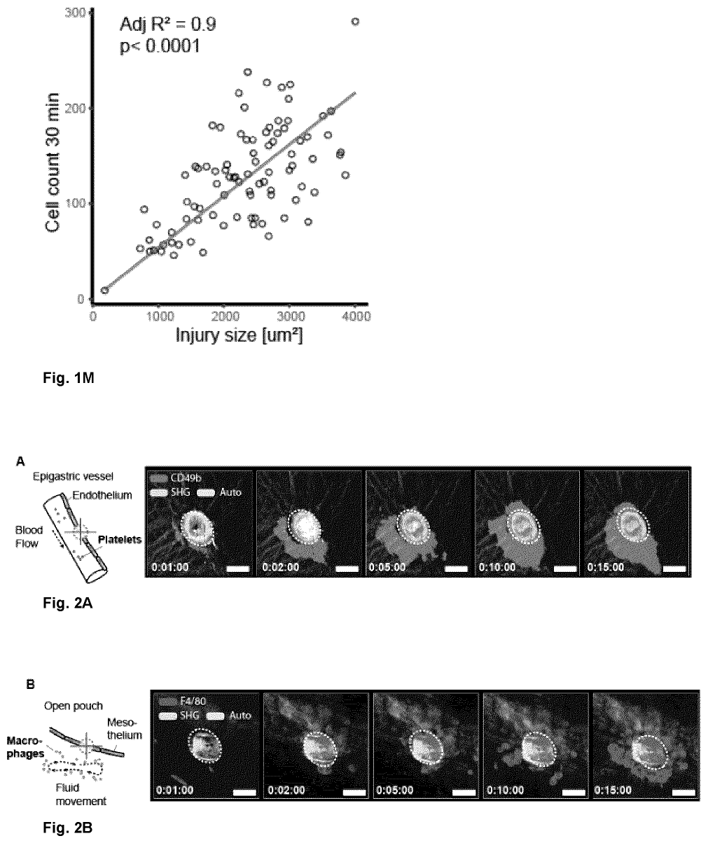

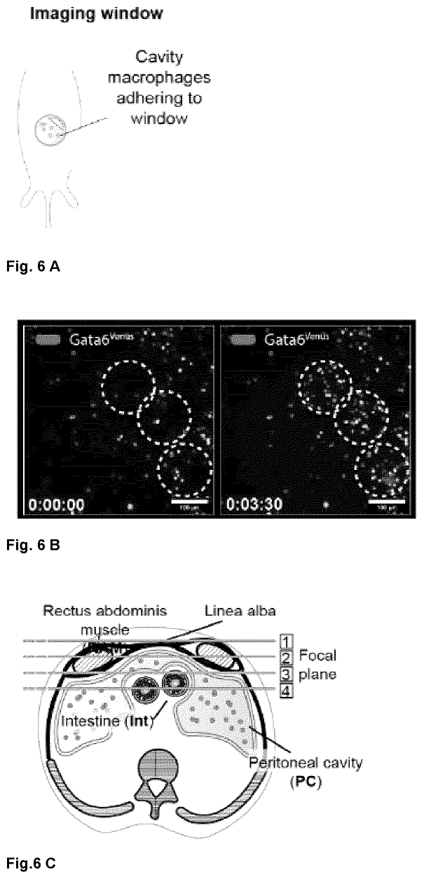

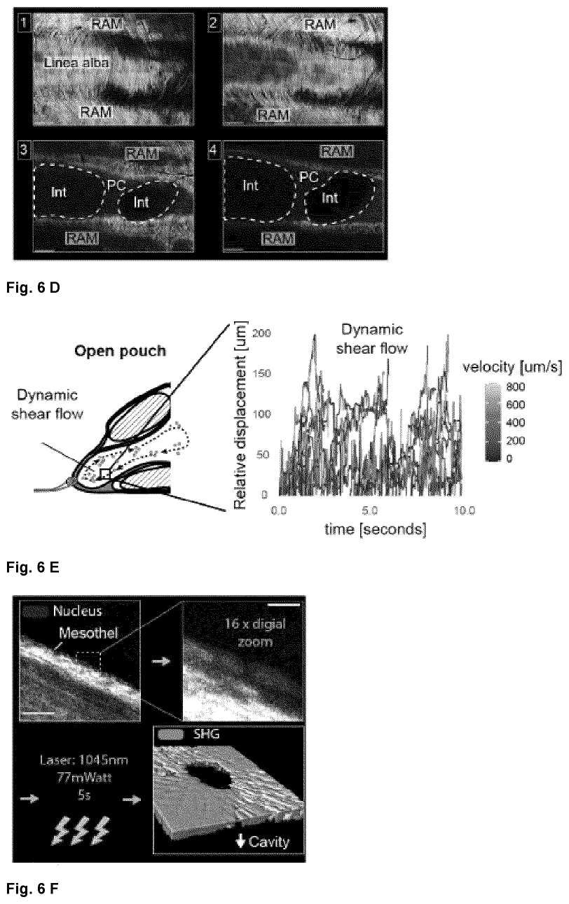

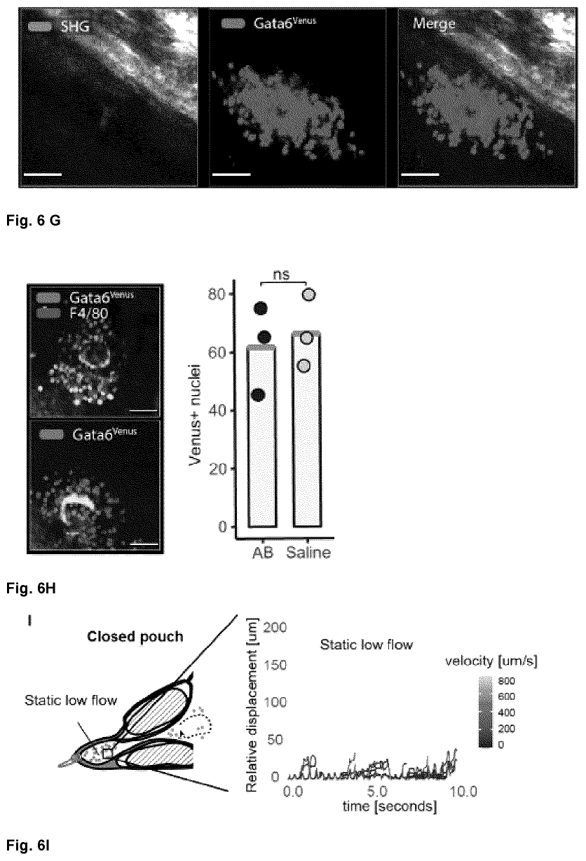

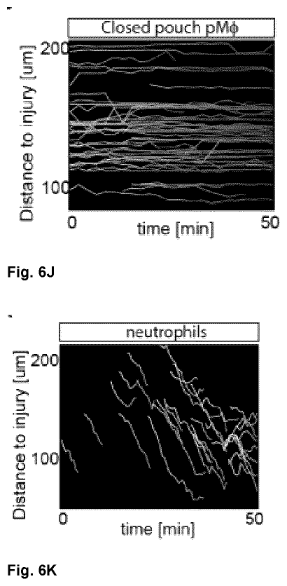

To study the migration of Gata6⁺ cavity macrophages in response to injury, we established a mouse intravital microscopy model. We found that any perturbation, including opening of the peritoneal cavity, or introducing an imaging window, was sufficient to alter the biology of these cells. Indeed, within minutes of placing an imaging window into the cavity, GATA6⁺ macrophages rapidly covered the foreign surface (FIGS. 6, A and B). To bypass this window artefact, we used multiphoton microscopy and extremely sensitive hybrid detectors to image the peritoneal cavity through the intact abdominal wall. The midline zone separating the recti abdomini muscles (Linea alba) provided a natural imaging window (FIGS. 1, A and B; FIGS. 6, C and D). The abdominal wall was immobilized by the formation of an anchored pouch (FIGS. 1, B and C). The pouch remained open to the remainder of the peritoneal cavity such that convection of peritoneal fluid was maintained, and organs could move within the space. Only the abdominal wall was immobilized. This model—referred to as the “open-pouch model” (FIG. 1D)—preserved the dynamics of peritoneal fluid motion (FIG. 6E). Fast (8 kHz) resonant scanning enabled the tracking of the rapidly moving peritoneal macrophages, which passively traversed the peritoneal cavity in a respiration-dependent and seemingly random pattern (FIG. 6E) with speeds of up to 800 μm/s (FIG. 6E). This observation accurately represented what we observed in the whole peritoneum. However, motion artefacts prevented continuous imaging outside the pouch.

These GATA6⁺ macrophages were free floating in the peritoneal fluid, were highly mobile, and differed from the GATA6⁻ macrophages embedded in the muscular abdominal wall, a compartment that is well separated from the peritoneal cavity by the mesothelium. These abdominal wall macrophages are sessile and capable of cloaking a single dead cell (but not more) after laser injury with their dendrites to prevent neutrophil-driven inflammation (13). Here we studied the reaction of the free floating GATA6⁺ cavity macrophages to an injury of the peritoneal wall. The induction of a focal thermal injury using a multiphoton laser allowed us to precisely position the lesion so that it breached the mesothelial monolayer that compartmentalized the fluid-filled peritoneal cavity and also affected the most superficial layer of the muscular abdominal wall (FIG. 6F). In the open-pouch model, the site of laser injury was covered with hundreds of cavity macrophages within minutes (FIGS. 1, D and E). Interestingly, cavity macrophages seemed to form tight multicellular aggregates at the injury site. Use of a Gata6^{Venus} reporter mouse (instead of anti-F4/80 antibody) demonstrated that this phenomenon was not caused by intraperitoneal fluid injection (FIG. 6G) nor was the aggregation increased by agglutination through intraperitoneal application of the F4/80 staining antibody (FIG. 6H). This allowed for the subsequent use of F4/80 staining in various knockout mice.

Recruitment of Gata6⁺ Cavity Macrophages

Because of the striking similarity to platelet recruitment, we queried whether macrophage recruitment required fluid shear flow. We constructed a peritoneal pouch and sealed it from the larger abdominal cavity (FIG. 1F), which eliminated the passive movement of cavity macrophages (FIG. 6I). Despite the abundance of macrophages within the sealed pouch, lack of flow through the pouch abrogated the aggregate formation of GATA6⁺ macrophages. In the closed-pouch model, significantly fewer cells were mobilized from suspension to the injury site confirming that cavity macrophages rely on passive transportation by dynamic flow of the peritoneal fluid for their recruitment (FIGS. 1, F and G; FIG. 6J).

The cavity macrophage recruitment was strikingly different from the canonical recruitment of neutrophils, which exited blood vessels and crawled through the interstitial tissue in a directed manner to the site of injured abdominal wall (FIGS. 1, H and I; FIG. 6K). Moreover, the recruitment time was much slower for neutrophils (>40 min) than for cavity macrophages (1-15 min).

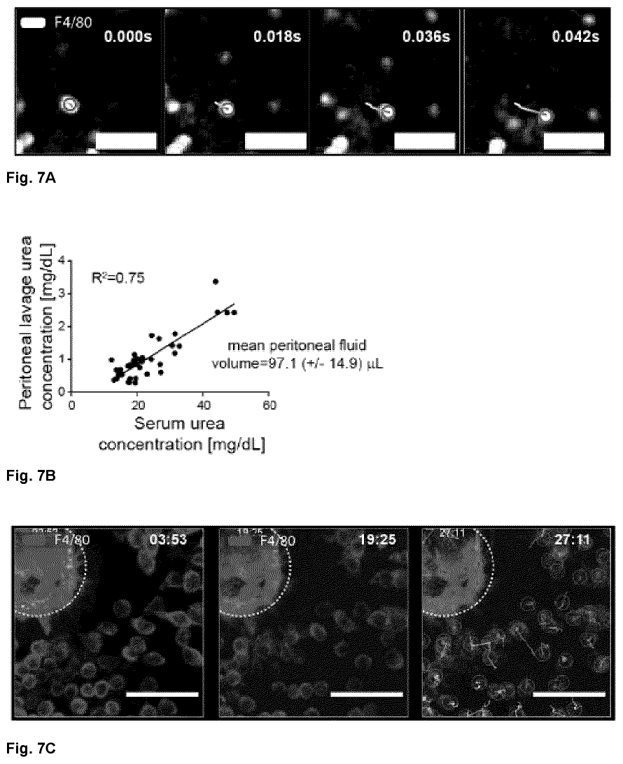

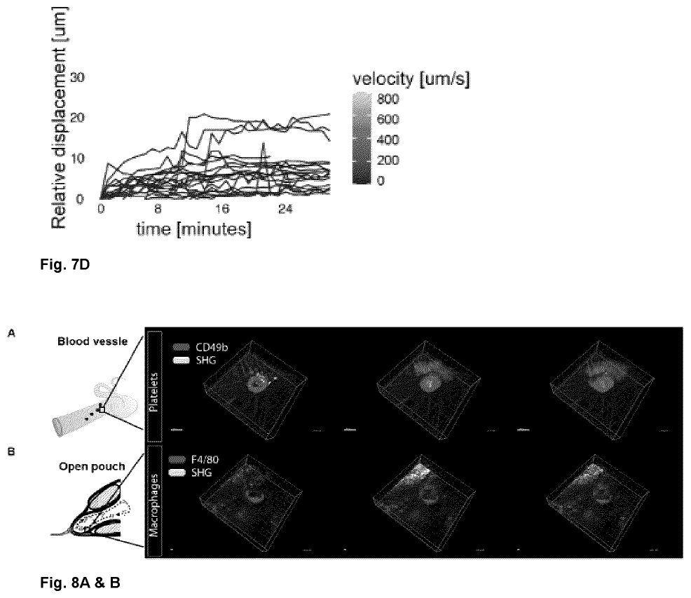

Using resonant scanning mode, we tracked cells in the fluid phase (FIG. 7A). Macrophages in the fluid phase showed a round appearance (FIGS. 1, J and K). However, upon contact, the macrophages adopted an elongated epithelioid phenotype (FIG. 1K and FIG. 7B) and formed stable adhesions that were able to resist high fluid shear stress of up to 8 dyn/cm² (or 0.8 Pa) based on a velocity of 800 μm/s (FIG. 6E) and an estimated viscosity of the peritoneal fluid approaching 1 Ps·s. Mean total peritoneal fluid volume of untreated mice was estimated to be 97±15 (SD) μl based on the known concentration of urea (FIG. 7B) yet no cells appeared to be resting on the mesothelium under basal conditions. Upon injury, a few macrophages tethered to the wounded area with subsequent macrophages tethering directly to already attached macrophages (secondary tethers). Although some cells crawled, the net displacement was less than 1-2 cell lengths over the whole imaging period (FIGS. 7, C and D), suggesting the initial tether and not chemotaxis was the primary mode of localization to the injury site. At the injury, the macrophage cell-cell aggregates covered the induced breach of the mesothelium (FIG. 1L) and the aggregates of cavity macrophages did not grow indefinitely suggesting a regulatory mechanism that controlled aggregate size. There was a strong correlation between damage size and number of cells attached after 30 min (FIG. 1M). Thus, the recruitment of macrophages is a regulated two-step process. First, free-floating macrophages tether to the injury and then additional macrophages form secondary tethers to these already attached macrophages. No obvious chemotactic behavior could be seen at either step of this recruitment process.

Gata6⁺ Macrophages Resemble Extravascular Platelets

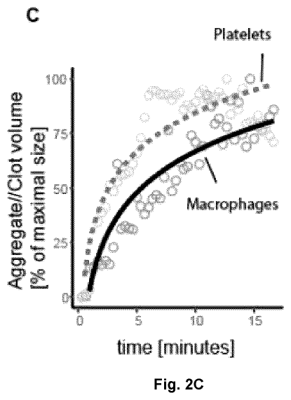

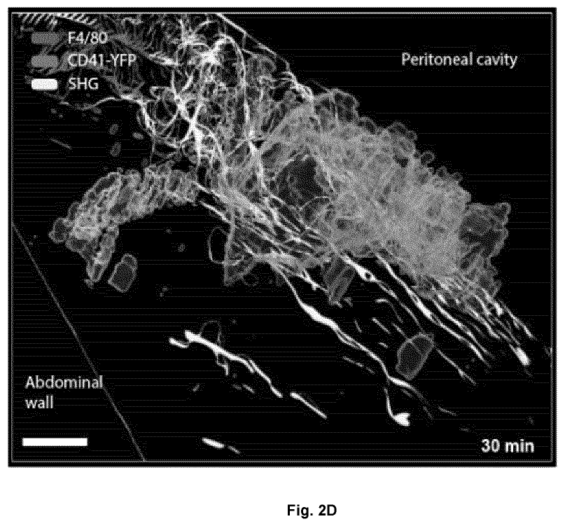

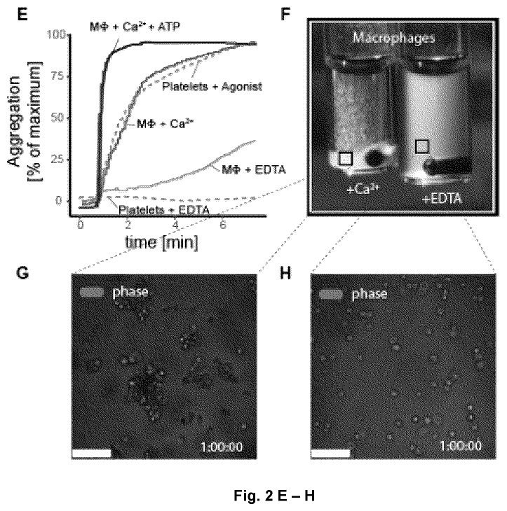

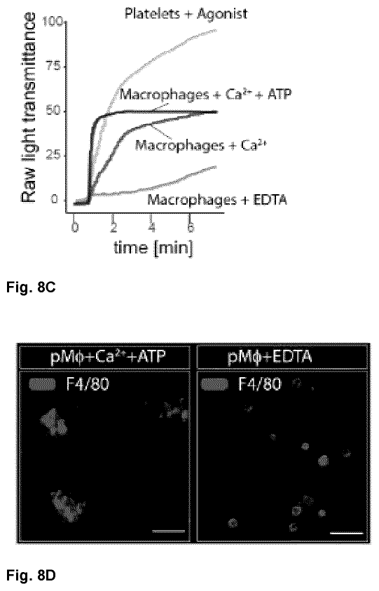

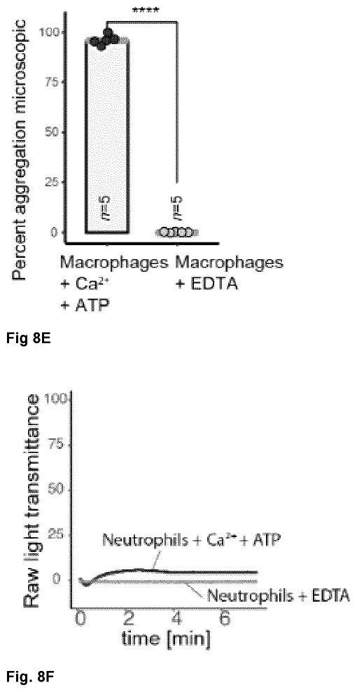

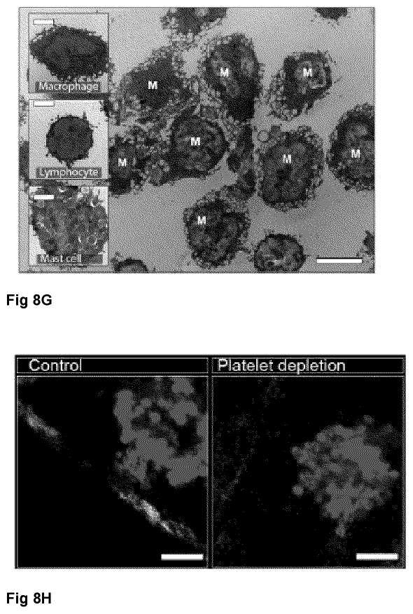

The ability to rapidly form aggregates under shear at injury sites was reminiscent of platelet aggregation and thrombus formation after damage of the vessel wall. For comparison, we stained platelets with fluorescently labeled anti-CD49b antibody and induced the same thermal laser injury to the epigastric vessel wall. A transendothelial injury elicited an intravascular platelet aggregation, which highly resembled cavity macrophage aggregation after transmesothelial injury (FIG. 2, A to C and FIGS. 8, A and B). For a direct head-to-head comparison, Itga2b^{YFP} (CD41) reporter mice in which platelets constitutively express fluorescent YFP were tracked together with intraperitoneal macrophages. Induction of a thermal injury spanning the vasculature (transendothelial) and peritoneal wall (transmesothelial) led to an intravascular platelet thrombus formation on one side of the injury, and the formation of a macrophage aggregate on the other side (FIG. 2D). However, at no stage did the platelets and macrophages intermix retaining localization in their respective compartments. Moreover, depletion of platelets did not affect the formation of the macrophage aggregate (FIG. 8H) suggesting no cross talk. Nevertheless, the kinetics were remarkably similar and both platelet and macrophage aggregates reached a stable size after 15 min (FIG. 2C).

In echinoids, coelomocyte aggregation occurs spontaneously when macrophages are removed from the coelom (7, 8). To probe the intrinsic aggregation capacity of peritoneal cavity macrophages, we used aggregometry, a method commonly used to assess platelet function in patients with bleeding disorders. Very much like coelomocytes, cavity macrophages formed spontaneous aggregates independent of serum or peritoneal fluid, a process that could be inhibited with EDTA (FIG. 2, E to H and FIG. 8, C to E). We were able to accelerate macrophage aggregation in vitro using adenosine triphosphate, an established activator of platelet aggregation (FIG. 2, E to H). The time needed to reach maximal aggregation under shear was the same as the time needed for platelet aggregation (FIGS. 2, E and G and FIG. 8, C to E). For comparison, we isolated blood neutrophils and subjected them to aggregometry and using identical conditions as for macrophages, they showed no tendency to aggregate (FIG. 8F). Furthermore, despite ample lymphocytes and mast cells within the peritoneal cavity, aggregates consisted only of macrophages when examined with electron microscopy (FIG. 8G).

The Molecular Mechanism for Macrophage Aggregation

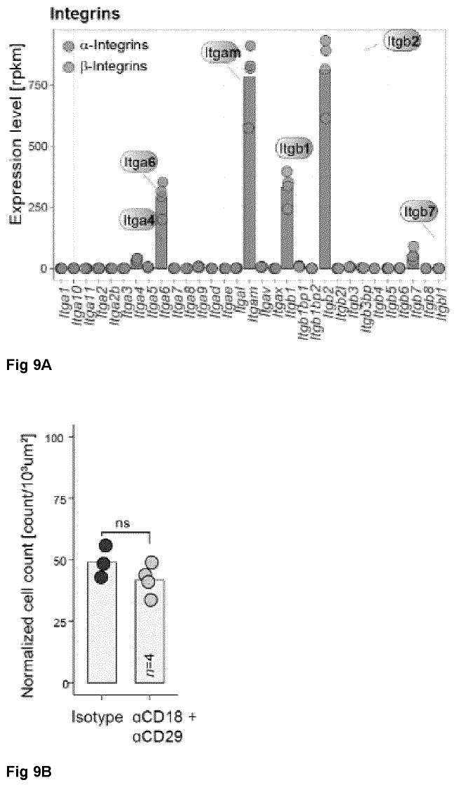

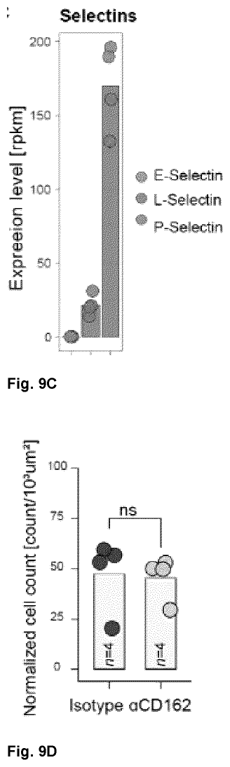

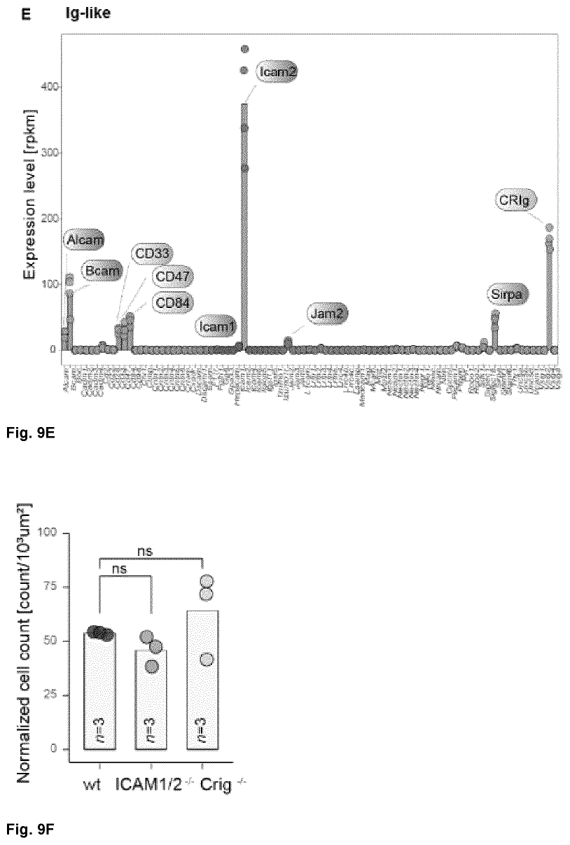

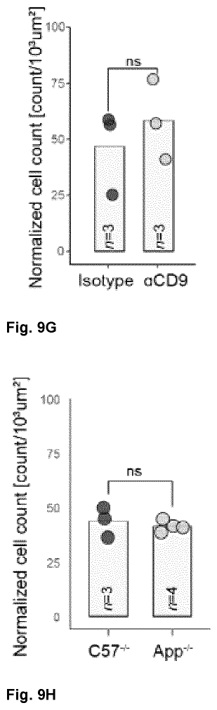

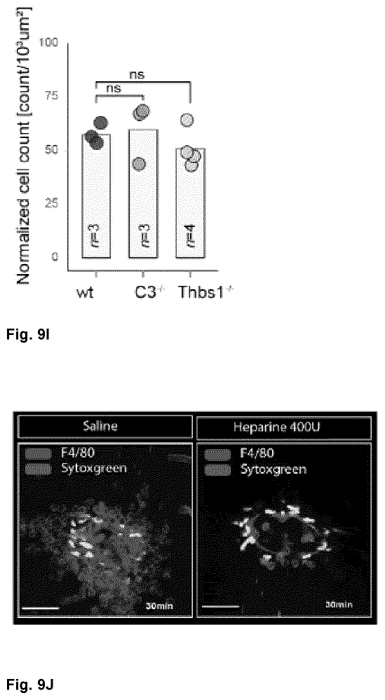

Next, we asked what controlled cavity macrophage adhesion and cluster formation to the injury site in vivo. As the observed cell accumulation occurred within minutes, we hypothesized that the responsible adhesion molecules were constitutively expressed by cavity macrophages. We probed a transcriptomic profile of cavity macrophages, recently published by our group (14), for expression of canonical adhesion molecules such as integrins, selectins, and Ig-like adhesion molecules. Cavity macrophages almost exclusively expressed integrin dimers containing either the β1 or β2 chain but not prototypical platelet integrins such as α2bβ3 (FIG. 9A). Combining blocking antibodies against β1 (CD29) and β2 (CD18) integrins did not result in a reduction of adhering cells after 30 min (FIG. 9B). Similarly, blocking PSGL (CD161), the ligand of both selectins expressed by cavity macrophages (P and L-selectin, FIG. 9C), had no effect (FIG. 9D). We also tested the most abundantly expressed Ig-like adhesion molecules, Icam1/2 and CRIg (FIG. 9E). Neither Icam1Icam2 double-knockouts nor Vsig4 (CRIg) knockouts showed impaired aggregation (FIG. 9F). We next investigated expressed genes listed under the gene ontology term “adhesion”. The resulting list of candidate molecules was further filtered for proteins localized on the plasma membrane of peritoneal macrophages. In addition to the aforementioned proteins, this approach uncovered the following targetable molecules: CD9, amyloid precursor protein (APP) and thrombospondin 1 (THBS1). APP has previously been reported to mediate neuronal adhesion, whereas THBS1 causes platelet adhesion. However, antibody blockade or genetic deletion of these molecules did not show any significant reduction in the number of macrophages at the injury site when compared with the respective wild-type or isotype controls (FIG. 9, G to I).

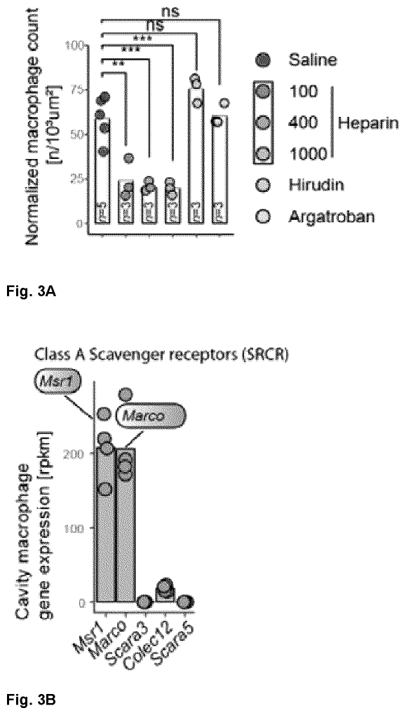

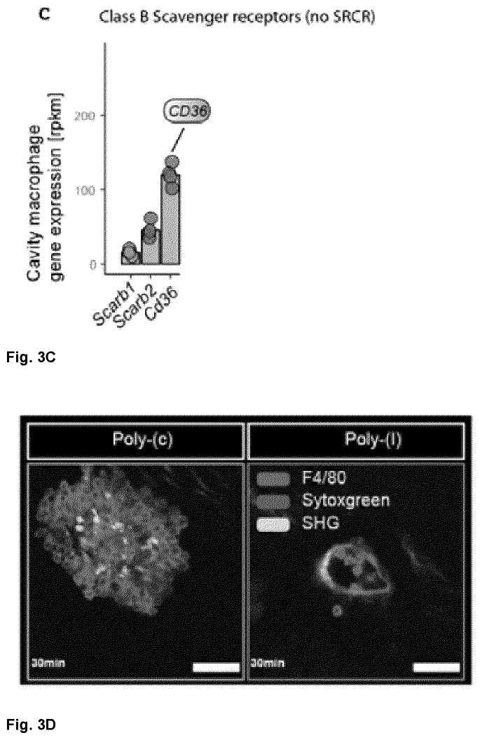

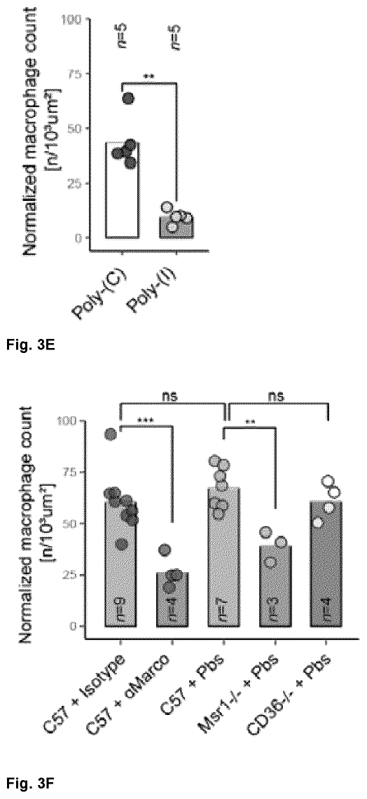

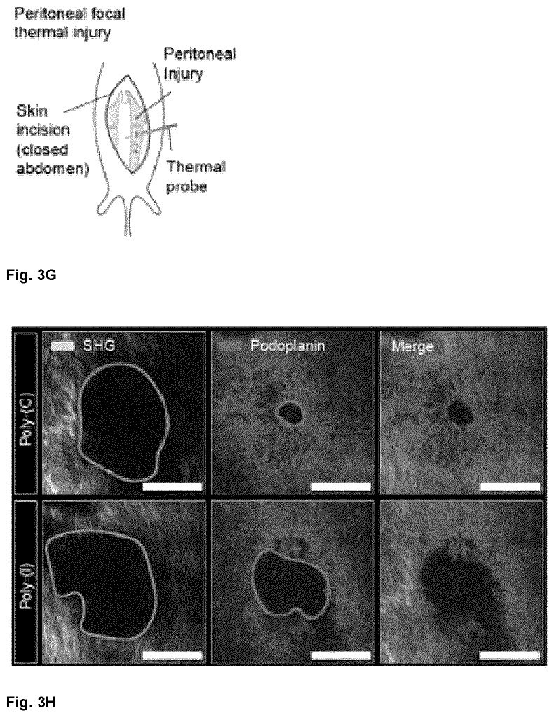

We then hypothesized that molecules not canonically associated with cell-cell adhesion may be involved. Previous reports demonstrated that intraperitoneal administration of heparin was able to inhibit the macrophage disappearance reaction in response to either an intraperitoneal hypersensitivity reaction (15) or infectious agents like Escherichia coli (11). In our system, intraperitoneal heparin injection significantly reduced the number of attaching cells to the sterile injury (FIG. 3A and FIG. 9J). However, unlike in the aforementioned studies, the use of direct thrombin inhibitors hirudin and argatroban did not replicate this effect (FIG. 3A). Therefore, we tested for a thrombin-independent activity of heparin. First, we investigated a role of the alternative pathway of the complement system, which has been described to be inhibited by heparin (16). However, C3-knockout mice did not show a difference compared to wild-type mice (FIG. 91). Heparin is a large polyanion, and as such, has many thrombin-independent anti-adhesive properties. Thus, we hypothesized that heparin inhibited macrophage adhesion and aggregation by neutralizing receptors that recognize charged motifs.

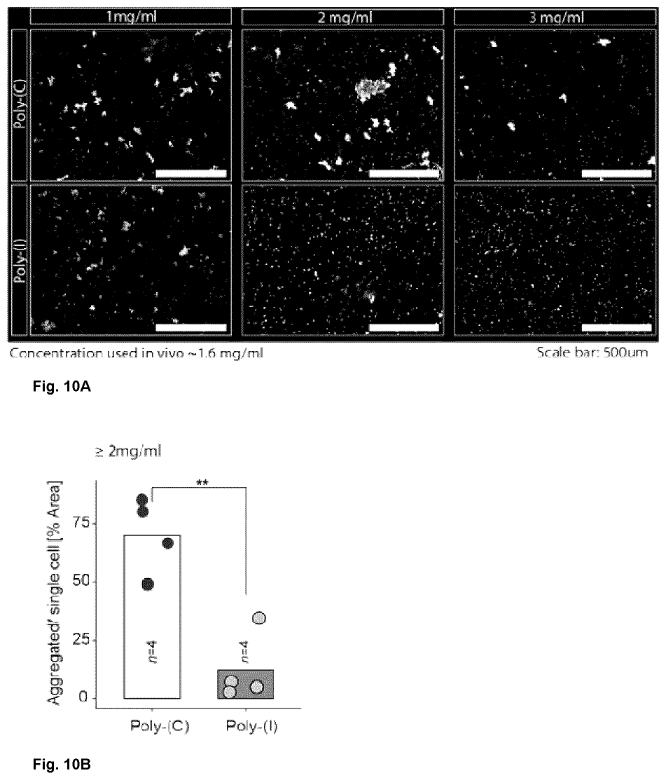

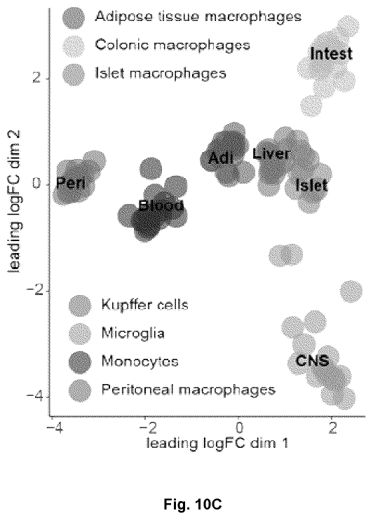

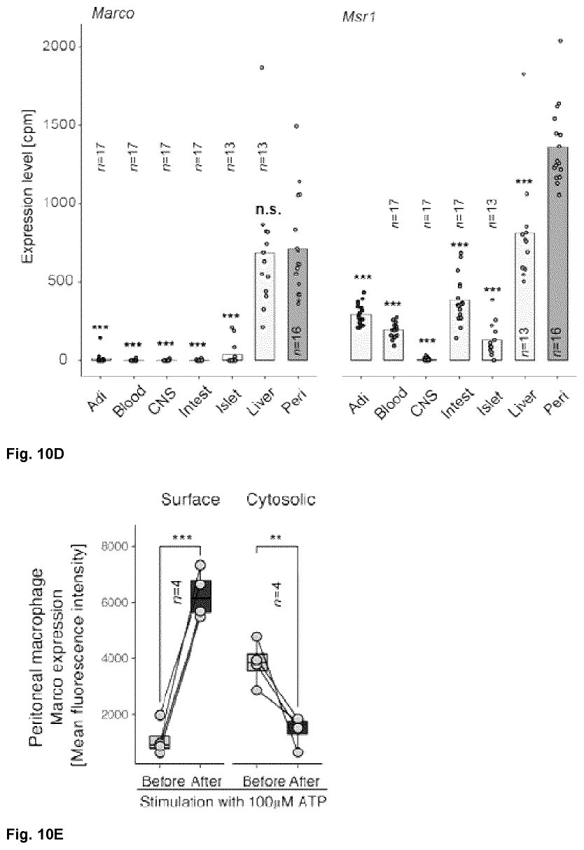

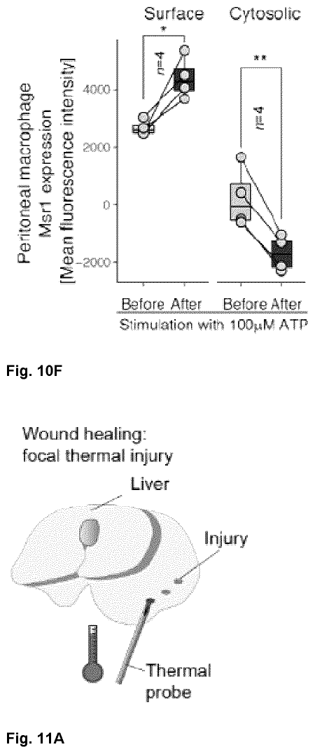

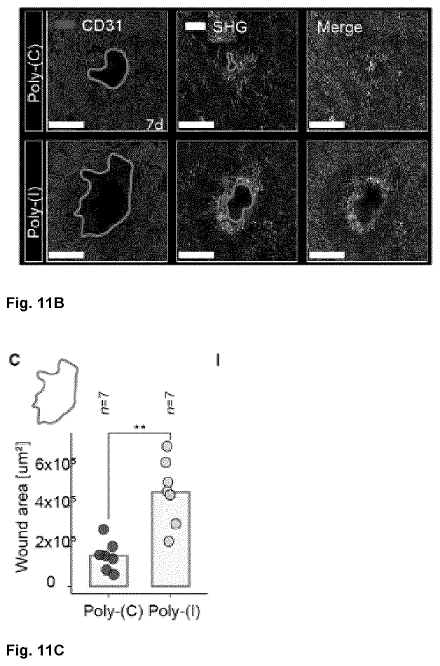

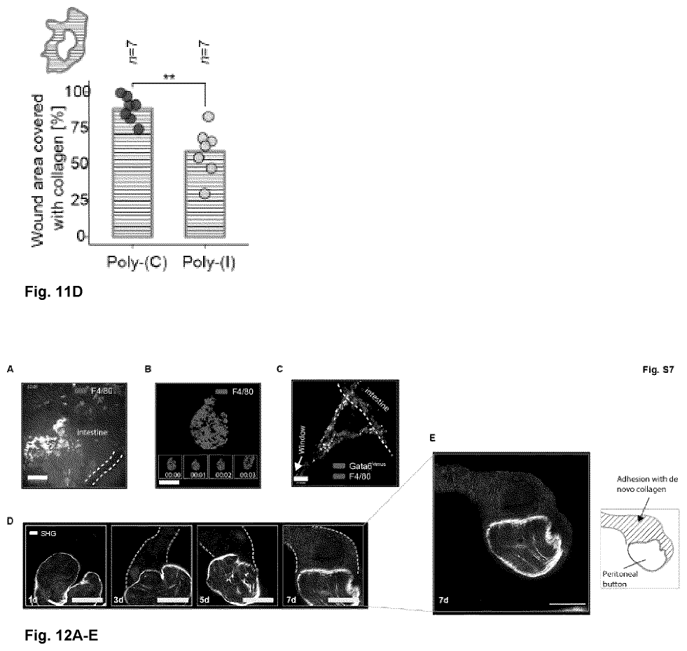

Murine scavenger receptors initially identified as macrophage receptors that recognize acetylated low-density lipoprotein (17) contain scavenger receptor cysteine-rich (SRCR) domains and promiscuously bind a wide variety of polyanionic ligands numbering in the hundreds (18). An SRCR superfamily protein was discovered as a cell-cell adhesion molecule in sea sponges (19) and an estimated 1,200 SRCR containing homologues are expressed by sea urchin coelomocytes (4, 20-22). Murine cavity macrophages express four SRCR containing murine homologues of which two are classified as class A scavenger receptors: the macrophage scavenger receptor 1 (MSR1) and the macrophage receptor with collagenous structure (MARCO) (FIG. 3B). For comparison, we examined CD36, a class B scavenger receptor without SRCR domains that is expressed at similar levels by murine cavity macrophages (FIG. 3C). Although Cd36-deficient mice exhibited normal aggregation of macrophages (FIG. 3F), blocking scavenger receptors with the negatively charged molecule polyinosinic acid (poly(I)) significantly decreased macrophage aggregation at the injury (FIGS. 3, D and E). Polycytidylic acid (poly(C)) served as a control. Furthermore, specific targeting of MARCO or Msr1 reduced the number of aggregating cells significantly when compared with their respective controls (FIG. 3F). To determine whether these receptors mediated the initial attachment to the wound or aggregation of the macrophages we used in vitro aggregometry to directly examine the latter. Poly(I) was able to prevent macrophage aggregation in vitro (FIGS. 10, A and B). Next, using our intravital imaging setup, we examined aggregate formation over time following poly(I) administration and observed a significant decrease in the macrophage-macrophage aggregate formation while the initial adhesion to the wound was left relatively intact (FIG. 3D). Thus, scavenger receptors in this system are not required for the initial (cell-matrix) attachment to the wound. Rather, they serve as secondary tethers during the second step, macrophage (cell-cell) aggregation.

A comparison of Marco and Msr1 transcriptomic expression across many macrophage populations in a previously published (23) data set revealed Marco expression exclusively in peritoneal and liver macrophages but not adipose tissue, colon, pancreatic islets, brain, or blood monocytes (FIG. 10C). Msr1 was expressed in most populations of macrophages, but expression in peritoneal macrophages was significantly higher than that of other populations studied (FIG. 10D). In addition to detecting these receptors on the surface of the peritoneal GATA6⁺ macrophages, activation of these cells with ATP resulted in a rapid (30 min) mobilization of MARCO and MSR1 from an intracellular pool to the membrane in vitro (FIGS. 10, E and F).

Next, we examined the functional consequences arising from the inhibition of cavity macrophage aggregation. A focal thermal injury of the parietal peritoneum through an intact abdominal wall showed prolonged healing when macrophage aggregation was blocked with poly(I), suggesting that the aggregate formation functionally aided the healing process (FIG. 3, G to J). Focal thermal lesions of organs within the peritoneum such as liver with minimal opening of the cavity were similarly affected (FIG. 10, A to D). In animals that did not receive poly(I), no lesions on the liver or abdominal wall were observed after 14 days and healing occurred without intra-abdominal scars, demonstrating the capacity to fully heal these lesions without a scarring phenotype.

Pathologic Scarring Mediated by Gata6⁺ Macrophages

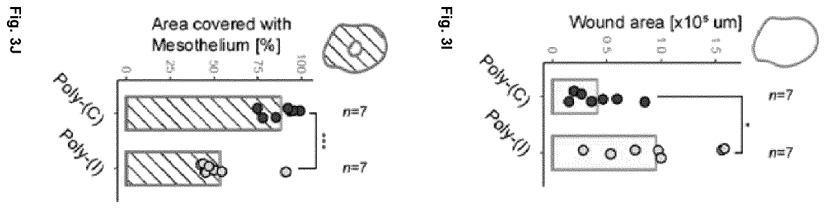

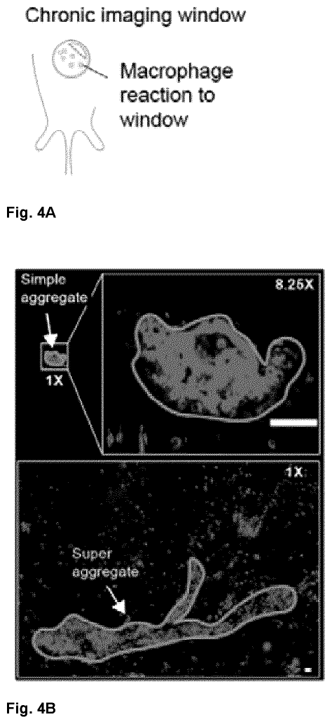

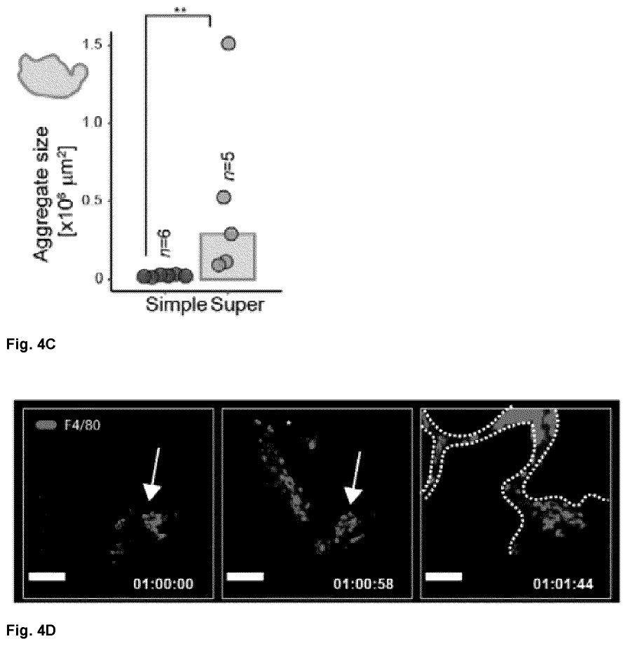

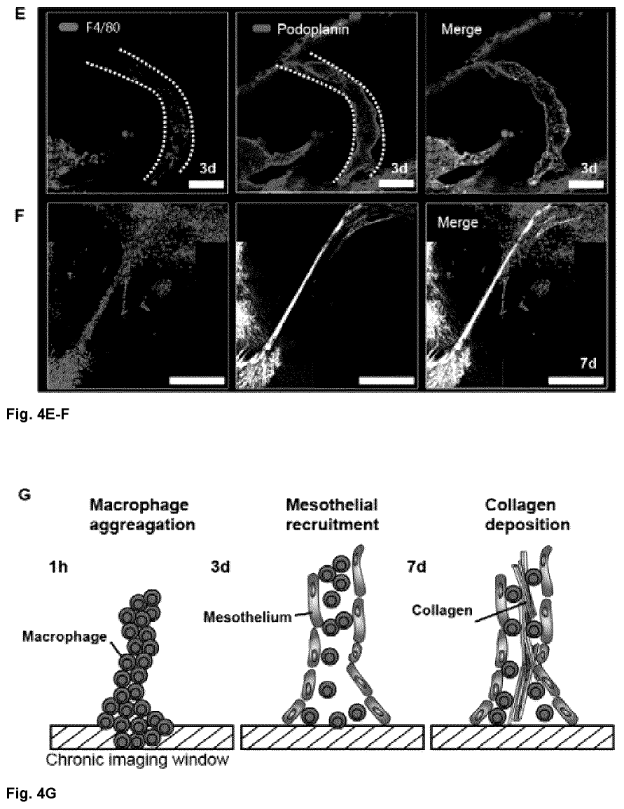

In echinoderms, the response to body wall injury includes the formation of an aggregate of coelomocytes and local production of collagen material (24, 25). In humans, intra-abdominal scars frequently occur after abdominal surgery manifesting as peritoneal adhesions. Peritoneal adhesions are defined as irreversible bands of scar tissue that attach abdominal structures at non-anatomic locations (26). Peritoneal adhesions are a major health burden for patients, leading to potentially life-threatening intestinal occlusion (26, 28, 29) and more than 300,000 additional abdominal operations per year in the US alone costing several billion dollars annually (27). In addition, peritoneal adhesions remain an unresolved clinical challenge which to date lack effective treatment. We therefore investigated whether the aggregation of mammalian cavity macrophages in response to injury could lead to the formation of peritoneal adhesions. Using a chronic imaging window that required the opening of the peritoneal cavity (FIG. 4A), we observed that macrophages aggregated within minutes on either the window surface (FIG. 6A) or the surface of adjacent abdominal organs (FIG. 12A) or formed free-floating aggregates (FIG. 12B). Subsequently, macrophage aggregates began to merge and form super-aggregates. The super-aggregates that formed in response to implanting an imaging window were up to 100 times the size of the simple aggregates that formed in response to peritoneal laser injury (FIGS. 4, B and C). Super-aggregates began to form bridges between the imaging window and intra-abdominal organs like the omentum (FIG. 4D) or the small intestine (FIG. 12C). Macrophage super-aggregates were covered with a mesothelial lining within 3 days (FIG. 4E). After 7 days, but not earlier, collagen was deposited within these macrophage super-aggregates (FIG. 4F and FIGS. 12, D and E). Both coverage with mesothelium and deposition of collagen are considered hallmarks of peritoneal adhesion formation (26). Thus, a severe iatrogenic disturbance of the peritoneal compartment, such as a laparotomy and introduction of a foreign body, can result in an exaggerated and dysregulated aggregation of cavity macrophages. The resulting super-aggregates are then precursors of peritoneal adhesions.

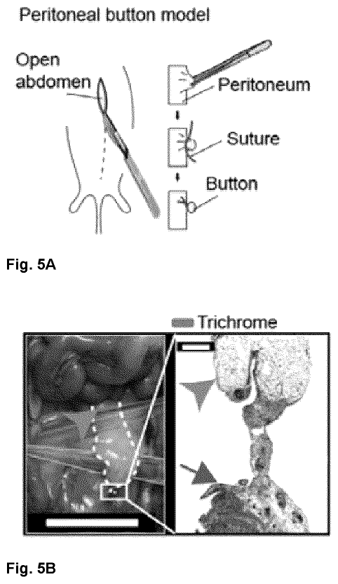

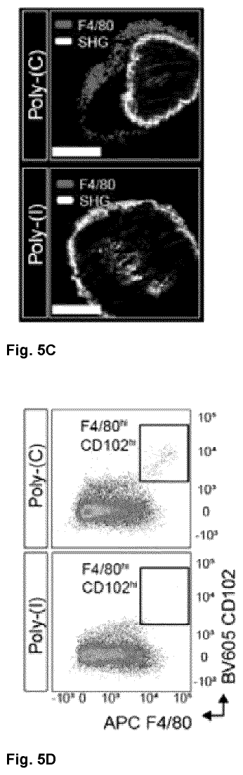

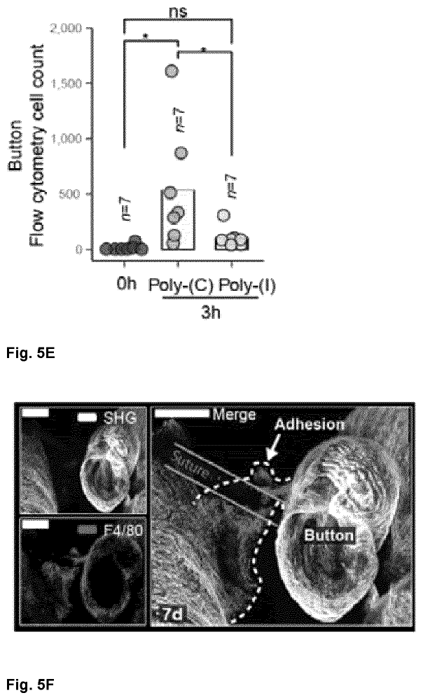

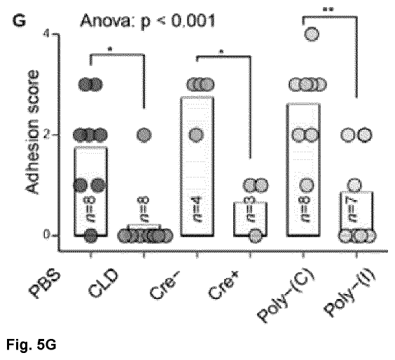

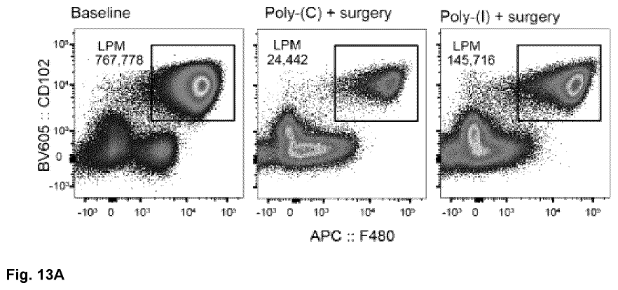

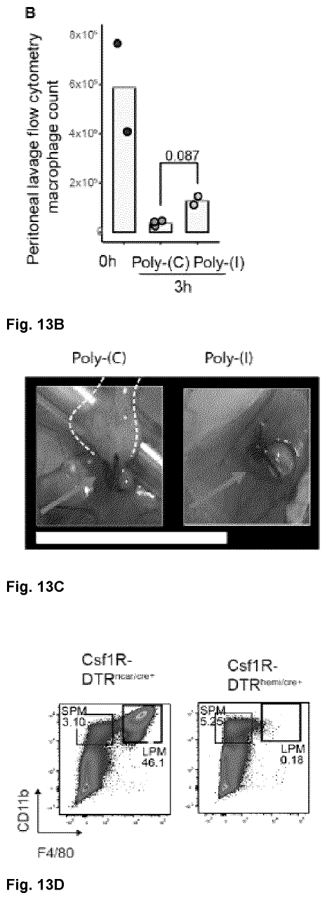

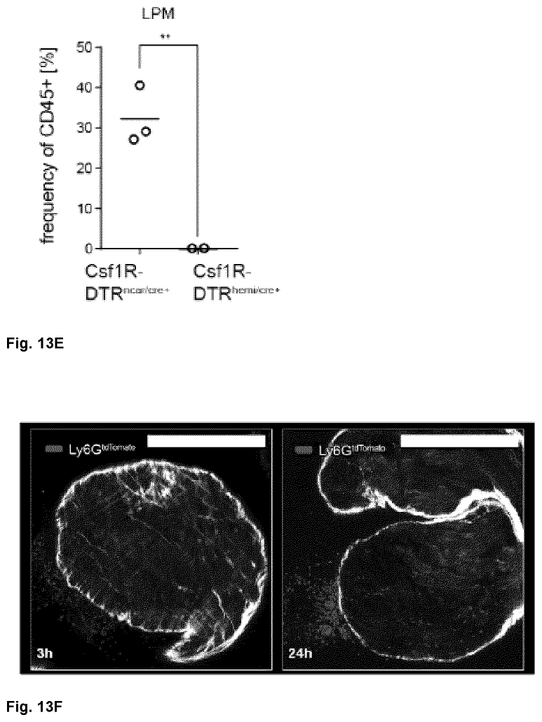

To quantify peritoneal adhesion formation in a system with more clinical relevance, we used a surgical sterile injury model that was previously described and referred to as the peritoneal-button model (30, 31). In this model, a laparotomy (midline) is generated and a portion of the peritoneal wall-including both the muscle layer and peritoneal membrane—is grasped with forceps and ligated at its base creating a button in each quadrant of the peritoneal cavity (FIG. 5A). In comparison to the focal thermal injuries, which heal without scar formation, this model provides a more iatrogenic setting with laparotomy, creation of ischemic pockets, and the introduction of foreign material (polypropylene suture). The model led to the reproducible formation of peritoneal adhesions (scars) between the buttons and adjacent intraabdominal organs within 7 days (FIG. 5B). Within 3 hours post-surgery, aggregates of cavity macrophages were recruited from the peritoneal fluid to the buttons (FIG. 5, C to E), a process that could be reduced when scavenger receptors were blocked with poly(I) (FIG. 5, C to E). Significant number of cavity macrophages engaged in this process as their numbers were greatly reduced in the peritoneal lavage after surgery, a process that was only partially inhibited with poly(I) (FIGS. 13, A and B). Interestingly, peritoneal adhesions harvested at 7 days post-surgery often formed at the sutures used to create the peritoneal buttons (FIG. 5F) and were full of macrophages with the highest concentration around the polypropylene sutures (FIG. 5F). The number and tenacity of peritoneal adhesions that formed within 7 days post-surgery was significantly reduced after both depletion of cavity macrophages and blocking macrophage recruitment by blocking scavenger receptors with poly(I) (FIG. 4G). Notably, neutrophils were also recruited to the peritoneal buttons after 4 hours (FIG. 12F), reflecting their response to injury. However, the depletion of neutrophils had no effect on adhesion formation (FIG. 12G).

### DISCUSSION

Primitive organisms such as the sea urchin are entirely dependent upon innate immunity to deal with pathogens and tissue injury. Indeed, the coelomocytes in these organisms appear to perform antimicrobial immunocyte functions but also rapidly “plug up” punctures that would otherwise fatally disrupt homeostasis. We show herein that mammals have retained these primordial immune cells with hybrid platelet-macrophage properties to ensure extremely rapid healing responses. Although numerous groups have reported that platelets retain some immune function (32-35), we now report that the GATA6⁺ macrophages-likely in all cavities-retain important platelet-like functions. Integrins mediate most adhesion processes in immune cells. However, the majority of these molecules, with the exception of platelet integrins, fail to function under high-shear conditions (36). The class A scavenger receptors are characterized by an evolutionarily conserved scavenger receptor cysteine-rich (SRCR) domain, a motif of 90-110 amino acids with several cysteine residues (37), which mediate charge interactions. They are thought to play critical roles in organisms such as the purple sea urchin, which possesses over 1200 SRCR domains (22). Interestingly, the SRCR superfamily is highly conserved in vertebrates (20, 21). For example, the SRCR domain of Msr1 (SR-A) is 78% conserved between mammalians and Xenopus tropicalis (western clawed frog) (21). Within the SRCR domain of Marco, the RGRAEVYY motif is highly conserved across mammals (21). To date, these receptors have been considered to play critical scavenging functions. In this work, we describe an important function of scavenger receptors in primordial GATA6⁺ macrophages: a critical role in macrophage aggregation as part of a recruitment pathway leading to repair of focal peritoneal injuries. It is likely that the scavenger receptors on each of two macrophages binds the same negatively charged polyanion ranging from chemically modified lipoproteins, acidic phospholipids, numerous polysaccharides, and polyribonucleotides to name a few that then bridge the cells as secondary tethers. We hypothesize that these focal types of perturbations reflect a type of injury that the immune system has been challenged with throughout evolution. However, iatrogenic procedures such as abdominal surgery, which include exposure to the external environment and potential implantation of foreign material, reflect a type of injury that appears to have no evolutionary precedent (38). In this scenario, peritoneal macrophages may cause detrimental scarring in an inadvertent attempt to tissue repair ad integrum. Thus, the inhibition of macrophage scavenger receptors may provide a target to prevent scar formation after surgery in the peritoneal cavity and perhaps other cavities that contain these cells.

Materials and Methods

Mice

All mice were maintained on the C57BL/6 background. Gata6^{H2B-Venus/+} mice (39)(MGI Ref. ID: J:226600) were a gift from Dr. A. K. Hadjantonakis (Memorial Sloan Kettering, NY). LysM^{eGFPI+} mice (40) (MGI Ref. ID: J:63254) were a gift from Dr. T. Graf (Albert Einstein University, NY), Ly6G^{Cre-tdTomato} catchup mice (41) (MGI Ref. ID: J:227159) were kindly provided by Dr. S. Lacroix (Université Laval, QC, Canada). CD41^{YFP/+} mice (42)(MGI Ref. ID: J:123159) were a gift from Dr. K. McNagny, University of British Columbia, Vancouver, BC, Canada. Icam/2^{-/-} mice were a gift from Dr. R. Alon (Weizmann Institute of Science, Israel). Icam1/2^{-/-} mice were generated by mating Icam1^{-/-} (MGI Ref. ID: J: 18743) and Icam2^{-/-} (MGI Ref. ID: J:54748) mice. Crig^{-/-} mice (43)(MGI Ref. ID: J:138691) were a gift from Dr. M. van Lookeren Campagne (Genentech, San Franscisco, CA). Csf1R^{HBEGF/mCherry} mice (Jax: 024046) were crossed with LyzM^{Cre} (Jax: 004781) as previously described (44) generating Cre⁺ and Cre⁻ littermates. Wild-type C57BL/6, C3^{-/-} (Jax: 3641), Msr1^{-/-} (Jax: 006096), CD36^{-/-} (Jax: 019006), Thbs1^{-/-} (Jax: 006141) and App^{-/-} (Jax: 004133) were purchased from Jackson laboratories. Animals were maintained in a specific pathogen-free double-barrier unit at the University of Calgary Animal Resource Centre. Mice were fed autoclaved rodent feed and water ad libitum. Male and female mice with an age between 8 and 12 weeks were used for experiments. Experimental animal protocols used in this study were approved by the Health Science Animal Care Committee of the University of Calgary (Reference number AC19-0138) and were in compliance with the guidelines from the Canadian Council for Animal Care.

Antibodies and Reagents

The following monoclonal antibodies and reagents were used for intravital microscopy staining: PE-conjugated anti-mouse F4/80 (0.8 μg per mouse intraperitoneally, BM8, BioLegend); PE-conjugated anti-mouse CD49b (1.6 μg per mouse intravenously, HMα2, BD); Alexa Fluor 488-conjugated anti-mouse podoplanin (4 μg per mouse intraperitoneally, eBio8.1.1, eBioscience). Sytox green nucleic acid stain (0.2 μl per mouse intraperitoneally, 5 mM stock solution, Invitrogen).

The following neutralizing antibodies (versus isotype controls) and reagents were intraperitoneally tested for their effect on macrophage recruitment: anti-mouse CD18 (100 μg per mouse, GAME-46, BD Pharmingen)+anti-mouse CD29 (100 μg per mouse, HM β31-1, BD Pharmingen) versus isotype rat IgG1, κ (100 μg per mouse, BioXcell)+hamster IgG (100 μg per mouse, eBioscience); anti-mouse CD162 (100 μg per mouse, 4RA10, BD Pharmingen) versus isotype rat IgG1 (100 μg per mouse, eBioscience); anti-human CD9 (10 μg per mouse, ALB 6, Santa Cruz) versus isotype mouse IgG1 (BD Biosciences). Heparin (Sandoz) was diluted in sterile saline to reach final dose of 100-1000 U per mouse. Argatroban (Sigma-Aldrich) was dissolved in DMSO at 20 mg/ml and diluted with saline to reach a final concentration of 300 μg per mouse. Hirudin (Multiplate Hirudin Blood Tube, Roche) was dissolved in saline and injected intraperitoneally at a dose of 1 tube per mouse. Polyinosinic acid (poly(I)) and polycytidylic acid (poly(C)) were purchased as potassium salt (Sigma-Aldrich) and dissolved in double-distilled water according to manufacturer's instructions to 5 mg/ml and 20 mg/ml, respectively. Immediately before experiments, the poly(C) stock solution was diluted with double-distilled water to a final concentration of 5 mg/ml. Both poly(I) and poly(C) were then diluted with saline to reach a final dose of 250 μg per mouse. A total of 125 mg Anti-mouse Marco antibody (ED31, Biorad MCA1849) versus rat IgG1 Isotype control (eBRG1, eBioscience 16-4301-85) was injected intraperitoneally in a total volume of 140 μl PBS per mouse. A volume of 100 μl anti-mouse thrombocyte depletion serum (Cedarlane, CLAD31440) versus normal rabbit serum (Cedarlane, CLSD403) was injected intra venously 24 hours before imaging.

Antibodies, dyes, and their concentrations used for flow cytometric analysis of peritoneal lavage are summarized in table 1.

TABLE 1
Antibodies and dyes for flow cytometry

| | | | Fluorescence | Company (Catatolgue | |
|---|---|---|---|---|---|
| | Clone | Isotype | labelling | number) | Dilution |
| CD11b | M1/70 | Rat IgG2b, κ | PE-Cy7 | cBioscience (25-0112-82) | 1:100 |
| Ly6C | HK1.4 | Rat IgG2c, κ | PerCP-Cy5.5 | BioLegend (128012) | 1:200 |
| Ly6G | 1A8 | Rat IgG2a, κ | PE | BioLegend (127608) | 1:200 |
| Ly6G/Ly6C | RB6-8C5 | Rat IgG2b, κ | PerCP-Cy5.5 | cBioscience (45-5931-80) | 1:200 |
| CD3 | 145-2c11 | Hamster IgG | FITC | cBioscience (11-0031) | 1:100 |
| MHCII | M5/114.15.2 | Rat IgG2b, κ | APC-Cy7 | BioLegend (107628) | 1:100 |
| F4/80 | BM8 | Rat IgG2a, κ | APC | BioLegend (123122) | 1:100 |
| F4/80 | BM8 | Rat IgG2a, κ | FITC | cBioscience (11-4801-85) | 1:100 |
| CD102 | 3C4 | Rat IgG2a, κ | BV605 | BD Biosciences (740346) | 1:100 |
| CD45 | 30-F11 | Rat IgG2b, κ | BV510 | BioLegend (103137) | 1:100 |
| CD19 | cBio1D3 | Rat IgG2a, κ | cFluor 450 | cBioscience (48-0193-82) | 1:100 |
| Msr1 | 1F8C33 | Rat IgG2a | AF647 | BioLegend (154702) | 1:100 |
| | | | Labelling kit | Invitrogen (A20186) | |
| Marco | ED31 | Rat IgG1 | AF594 | Bio Rad (MCA1849) | 1:100 |
| | | | Labelling kit | Invitrogen (A10239) | |
| Viability | — | — | Ghost Dye | Tonbo Biosciences | 1:1000 |
| | | | Red 710 | | |
| Viability | — | — | Fixable far | Invitrogen (L34973) | 1:1000 |
| | | | red dead cell | | |
| | | | stain kit | | |

The following reagents were intraperitoneally injected at 24 hours before surgery for their effect on post-surgical adhesion formation: Diphtheria toxin from Corynebacterium diphtheriae (5 ng per gram of body weight, in Csf1r^{Cre} iDTR mice) or clodronate- or PBS-loaded liposomes (100 μl per mouse, clodronateliposomes.com).

Multiphoton Intravital Microscopy (IVM)

Mice used for IVM were 8-10 weeks old weighted an average of 20 g. Mice in each experiment were of the same sex. If not specified otherwise, mice were generally treated pharmacologically or with neutralizing antibodies 20 min before imaging. Mice were anesthetized with a mixture of ketamine hydrochloride (200 mg/kg, Rogar/SBT) and xylazine hydrochloride (10 mg/kg, MTC Pharmaceuticals). After anesthesia, the tail vein was cannulated for administration of additional anesthetic, antibodies, or other reagents in certain experiments.

The pouch model IVM (FIG. 1) was prepared with the mouse in supine position. Thermal support was given with a heated stage to maintain 37° C. body temperature. The fur and skin above the ventral abdominal wall were aseptically cleaned with 70% ethanol and then removed. Care was taken not to open the peritoneal cavity. If the peritoneal cavity was accidentally opened, the mouse was immediately euthanized. Staining antibody or dye was intraperitoneally injected with a 28G needle in midline about 10 mm above the symphysis. After withdrawing the needle, a suture was placed to seal the hole caused by the injection. This suture was then used to immobilize a part of the peritoneum onto the coverslip. For this, the mouse was placed in a semi-prone position. Placing a cotton tip with slight top to bottom pressure helped to further increase imaging stability yet preserved a connection between the imaging pouch and peritoneal cavity so that peritoneal fluid movement was preserved. The closed pouch was prepared by applying more pressure on the cotton tip which allowed us to separate the pouch from the peritoneal cavity in terms of fluid movement. After IVM, all mice were immediately euthanized.

Abdominal-wall-flap IVM was used to visualize the mesothelium and epigastric blood vessels (FIG. 2A, FIG. 3, F-I). Mice were placed in supine position on heated stage to maintain 37° C. body temperature. The fur and skin above the ventral abdominal wall were removed and then aseptically cleaned with 70% ethanol. Staining antibodies (anti-mouse podoplanin and anti-mouse F4/80 as described above) were injected. After mice were incubated for 10 min on a heated stage, the peritoneum was incised with an inverted U-shaped incision ranging from directly below the xyphoid process to the inguinal ligament on both sides. This created a perfused flap of peritoneum and the rectus abdominis muscles. The flap was washed with 1 ml of PBS and placed directly on the glass coverslip of the heated stage for imaging of abdominal wall or epigastric blood vessels.

Intravital liver imaging was performed as previously described (45). Exposed tissues were visualized with a Leica SP8 DIVE inverted microscope equipped with 4 Tune, a tunable in vivo detection system and a multiphoton light path. All images shown here were acquired using a HC FLUOTAR L 25×/0.95 W objective with water as immersion medium and 2× digital zoom. Fluorescent moieties were excited with a tunable InSight ×3 ultrafast laser (Spectra-Physics) at 800-1000 nm. Signal was detected using external, extremely sensitive non-descanned hybrid detectors. Second harmonic generation by collagen was recorded by tuning the detector to excitation wavelength divided by two ±20 nm. Leica LAS X software was used to drive the microscope.

In Vivo Interventions

For surgical procedures, mice were 8-12 weeks old with a body weight >20 g. Mice in each experiment were of the same sex. Mice were treated before surgery with poly(I), poly(C), liposomes, or diphtheria toxin as described above. Anesthesia was induced and maintained with isoflurane. Eye lubricant was topically applied and buprenorphine (0.1 mg/kg) was subcutaneously administered.

The peritoneal focal thermal injury was adapted from the focal thermal liver injury that was previously described (46). The skin was incised in midline. Care was taken not to injure the peritoneum or open the peritoneal cavity. The peritoneum was lifted in midline and three focal injuries were induced through the rectus abdominus muscle using the tip of a heated 30G needle mounted on an electrocautery device. Finally, the skin was sutured with 6-0 Prolene (Ethicon) running suture.

For the peritoneal button injuries, skin was incised in midline. The peritoneal cavity was accessed through midline laparotomy. Standardized lesions were induced as previously described (47). In brief, a small portion of the peritoneum was grasped and ligated at its base using 4-0 Prolene suture (Ethicon), creating a standardized peritoneal button. This was repeated for a total of four buttons, one in each quadrant. Finally, peritoneum and skin were sutured with 6-0 Prolene (Ethicon) running suture.

The abdominal imaging window (FIG. 6, FIG. 4) was manufactured and implanted as previously described (48). In brief, a round cover glass was mounted on a custom-made titanium ring and sterilized in Ethanol. A midline laparotomy was performed and a purse-string suture (5-0 Vicryl Rapide, Ethicon) was placed in a circular fashion along the whole incision. Then, the cover-glass titanium-ring was inserted, and the suture was tightened, securing the titanium ring in place and hermetically sealing the abdominal cavity from the exterior. Mice were monitored for weight loss and repetitively imaged for up to 7 days.

Focal thermal injury in the liver was performed as previously described (49). In brief, a mini-laparotomy was made just below the xyphoid to expose the liver. Three focal injuries were induced on the surface of the liver using the tip of a heated 30G needle mounted on an electrocautery device. The incision was sutured closed, and animals could recover for imaging of indicated time points after injury.

For bone marrow chimera generation, 6-week-old female C57BL/6J mice were lethally irradiated (2×525 cGY) and subsequently reconstituted with Gata6^{H2B-Venus} bone marrow cells for 8 weeks.

Peritoneal Fluid Volume Estimation

Urea nitrogen diffuses freely between the peritoneal cavity and the serum with a very fast equilibration time (50). Therefore, we assume that blood urea nitrogen [BUN] and peritoneal fluid urea nitrogen [PerUN] concentrations are in equilibrium: [BUN]˜[PerUN]. During harvesting, the peritoneal cavity was flushed with a known volume (V_{wash}), diluting the original peritoneal fluid of unknown volume (Vₚₑᵣ). Urea nitrogen concentration in the blood [BUN] and in the peritoneal lavage [WashUN] was determined using a calorimetric detection kit (Invitrogen, #EIABUN) according to the manufacturer's instructions. Based on the formula: [PerUN]×Vper=[WashUN]×Vwash, the peritoneal fluid volume was estimated, by substituting [PerUN] with [BUN]: Vper=Vwash×[WashUN]/[BUN].

Adhesion Scoring, Whole Mount Imaging, and Tissue Clearing

Prior to sacrifice, mice were anesthetized (10 mg/kg xylazine hydrochloride and 200 mg/kg ketamine hydrochloride). Laparotomies were performed in an inverted U-shape. Adhesion were scored based on their tensile strength and vascularization (Table 2) as previously described (51). The animal was subsequently euthanized and perfused with 2 mM EDTA and 4% PFA containing PBS. Peritoneal buttons/adhesions were excised and post-fixated in 4% PFA containing PBS for 2-4 hours. Tissue staining and clearing was performed as previously described (52). In brief, fixed biopsies were washed and permeabilized three times for 30 min in 1% Triton X containing PBS and subsequently blocked two times for 1 hour in 1% Triton, 10% FBS, and 0.02% sodium azide containing PBS. Then the biopsies were incubated with fluorescently conjugated monoclonal antibodies for 1-4 days at 4° C. on a rotation device. Samples were then washed three times for 1 hour in 1% Triton X containing PBS followed by sequential dehydration steps (50%, 70%, 99%, and 99% EtOH) for 4 hours at 4° C. After dehydration, the buttons were placed in ethyl cinnamate (Sigma). For imaging, cleared biopsies were placed in an ethyl cinnamate containing vessel with the suture facing downward. Image acquisition was performed using a Leica TCS SP8 inverted multiphoton laser-equipped microscope. All images shown here were acquired using a HC FLUOTAR L 25×/0.95 W objective with water as immersion medium. Fluorescent moieties were excited with a tunable InSight ×3 ultrafast laser (Spectra-Physics) at 800-1000 nm. Signal was detected using external, non-descanned hybrid detectors. Second harmonic generation by collagen was recorded by tuning the detector to excitation wavelength divided by two ±20 nm. Leica LAS X software was used to drive the microscope.

TABLE 2
Adhesion Score

| Grade | Description |
|---|---|
| 1 | Filmy and easy to separate or separates spontaneously by |
| | accessing the peritoneal cavity. |
| 2 | Blunt dissection possible, beginning vascularization but no |
| | visible* bleeding when separated. |
| 3 | Lysis possible by sharp dissection only, clear |
| | vascularization, visible* bleeding when separated. |
| 4 | Lysis possible by sharp dissection only, organs strongly |
| | attached with severe adhesions, damage of organs when |
| | separated. |

*For the current study, a surgical telescope with 2.5× magnification was used for all experiments.

Cell Isolation and Flow Cytometry

Mouse peritoneal lavage was performed in anesthetized animals by injection and aspiration of three times 5 ml of sterile ice-cold PBS containing 2 mM EDTA into the peritoneal cavity and subsequent retrieval. Lavage samples were subsequently centrifuged at 500 g for 5 min at 4° C. and the cell pellet was subsequently processed for flow cytometry. Peritoneal buttons were excised and subsequently digested in 125 U/ml collagenase IV (Worthington Biochemical), 60 U/ml DNasel (Roche) containing HBSS for 30 min in a 37° C. water bath with repetitive shaking. Homogenates were initially passed through a 70-μm cell strainer and spun down at 500 g for 5 min at 4° C. and cell pellet was directly processed for flow cytometry. The cells were incubated in protein based live/dead stain (Table 1) and blocked using anti-mouse CD16/32 antibody (2.4G2 clone, BioXcell) for 20 min. Cells were incubated for 20 min with fluorescently labeled antibodies (Table 1). Samples were run using BD FACS Canto flow cytometer and analyzed using FlowJo software (Tree Star). Gata6⁺ cavity macrophages were pre-gated on singlet, live, CD45⁺CD19⁻CD3⁻Ly6G⁻CD11b⁺ and then identified as F4/80^{hi}CD102⁺.

Activation of Peritoneal Macrophages

To study cell activation mouse peritoneal lavage was performed as described above. Cells were spun down and resuspended in RPMI medium (37° C.). Then, 100 μM ATP and 2 mM calcium (treatment group) or 2 mM calcium only (negative control) was added. Tubes containing cells were then incubated in the cell-culture incubator with the leads open for 30 min. The cells were then centrifuged at 500 g for 5 minutes and washed twice with cold PBS. Half of the samples were cells were permeabilized using the BD Fixation/Permeabilization Kit according to the manufacturer's instructions (BD Biosciences). Cells were incubated with fluorescently labeled antibodies (Table 1) and the appropriate FMO controls. Samples were run using BD FACS Canto flow cytometer and analyzed using FlowJo software (Tree Star). Gata6⁺ cavity macrophages were pre-gated on singlet, live, CD45⁺Ly6G/C⁻CD11b⁺ and mean fluorescence intensity of Msr1 and Marco for both permeabilized and unpermeabilized cells was measured on F4/80^{hi}CD102⁺ cells. Cytosolic expression was estimated by subtracting surface expression (non-permeabilized) from total expression (permeabilized).

Aggregometry

Macrophages were isolated by peritoneal lavage as described above with the use of 2 mM EDTA containing HBSS instead of PBS. Macrophages were centrifuged at 300 g for 5 min in a 50-ml polystyrene tube. The pellet (with approximately 100 μl of fluid remaining with 3×10⁶ cells) was diluted in HBSS containing 2 mM Ca²⁺ to a final volume of 400 μl. It was then transferred to a aggregometry cuvette, which was warmed to 37° C. and continuously stirred (400 rpm). Light transmission was recorded on a 700 Whole Blood/Optical Lumi-Aggregometer (Chrono-log) and was expressed in arbitrary units with buffer representing 100% transmission and washed macrophage suspension 0% transmission, respectively. Agonists and calcium were added to reach a Ca²⁺ concentration of 4 mM total or 2 mM free calcium in a buffer containing 2 mM EDTA. The preparation of platelet aggregometry was performed as previously described (53). Washed platelets (50 μl with 5×10⁵ platelets per microliter) were diluted into 110 μl of Tyrode-HEPES buffer containing 2 mM Ca²⁺ and 100 μg/ml human fibrinogen. Agonists (ATP, thrombospondin) were added at the indicated concentrations to the continuously stirred platelet suspension. Murine neutrophils were isolated from the bone marrow of wild-type C57Bl/6, mice as previously described (46). Briefly, bone marrow was collected from the femurs and tibias of euthanized mice, and neutrophils were isolated using a discontinuous Percoll gradient consisting of a Percoll solution (9 ml of Percoll and 1 ml 10× of HBSS) diluted to 72%, 64%, and 52% in 1×HBSS. Following centrifugation, the neutrophil band was removed and washed in HBSS. Cell pellets were resuspended in HBSS containing either 2 mM Ca2⁺ or EDTA. Aggregometry was performed as described above.

Analysis of RNA-Seq Data

The dataset used to probe gene expression or peritoneal cavity macrophages was previously published (14) and is accessible on the gene expression omnibus (GEO) database (GSE131724). In this study, peritoneal cavities of 8-week old C57BL/6 mice were lavaged. The F4/80^{hi} CD102⁺ cells were sorted and underwent bulk RNA sequencing. Data were demultiplexed and converted to FastQ using Bcl2fastq 2.17.1.14 (Illumina) and mapped to mm10 with Tophat 2.1.1. RPKM counting and normalization were performed using Partek GS 7.18.0723. Genes of interest were filtered and displayed for manually selected genes and Pathway gene sets from Gene Ontology (GO) biological processes (accessed at www.informatics.jax.org) using the R environment for statistical computing.

The dataset used to compare Scavenger receptor expression across macrophages isolated from different tissues was previously published (23) and was accessed on the GEO data base (GSE133127). Count data were analyzed as previously described (54). In brief, raw counts transformed were to log-counts-per-million using the calcNormFactors( ) and cpm( ) functions of the edgeR package, after filtering for genes that were not expressed using the function filterByExpr( ). Unsupervised clustering was done using the plotMDS( ) function of the limma package using normalized counts. Differential expression analysis was performed using linear modelling and subsequent Bayes moderation after removing heteroscedascity from count data using voom( ).

Image Analysis

Images were exported as uncompressed .tif files and analyzed using ImageJ (55). Tracking was generally performed in an automated fashion using the TrackMate plugin (56). The cell shown in FIG. 1J was tracked manually. Cell counts and injury size was measured manually in ImageJ. Three-dimensional visualizations and volume quantifications were performed in Imaris software. Analysis results from ImageJ and Imaris software were exported as .csv files and analyzed/plotted using R statistical environment.

Transmission Electron Microscopy

Aggregation of macrophages was performed as described above. Samples were submerged with fixative which was prepared as follows: 2.5% glutaraldehyde (Agar Scientific, Stansted, Essex, UK) in 0.15 M HEPES (Fluka, Buchs, Switzerland) with an osmolarity of 670 mOsm and adjusted to a pH of 7.35. The cells remained in the fixative at 4° C. for at least 24 hours before further processing. They were then washed with 0.15 M HEPES three times for 5 min, postfixed with 1% OsO4 (EMS, Hatfield, USA) in 0.1 M Na cacodylate buffer (Merck, Darmstadt, Germany) at 4° C. for 1 hour. Cells were then washed in 0.05 M maleic NaOH buffer three times for 5 min and dehydrated in 70, 80, and 96% ethanol (Alcosuisse, Switzerland) for 15 min each at room temperature. Subsequently, cells were immersed in 100% ethanol (Merck, Darmstadt, Germany) three times for 10 min, in acetone (Merck, Darmstadt, Germany) two times for 10 min, and finally in acetone-Epon (1:1) overnight at room temperature. The next day, cells were embedded in Epon (Sigma-Aldrich, Buchs, Switzerland) and left to harden at 60° C. for 5 days.

Sections were produced with an ultramicrotome UC6 (Leica Microsystems, Vienna, Austria): first semithin sections (1 μm) were used for light microscopy, which were stained with a solution of 0.5% toluidine blue O (Merck, Darmstadt, Germany); then ultrathin sections (70-80 nm) were used for electron microscopy. The sections were mounted on single slot copper grids and stained with UranyLess and lead citrate with an ultrostainer (Leica Microsystems, Vienna, Austria). Sections were then examined with a transmission electron microscope (Tecnai Spirit, FEI, Brno, Czech Republic) equipped with a digital camera (Veleta, Olympus, Soft Imaging System, Munster, Germany).

Design and Statistical Analysis

Sample size was determined prior to experiment for all experiments used for hypothesis testing (i.e. data that include statistical inference) except for RNA-Seq data sets that were re-analyzed.

Mice receiving pharmacological treatment were randomized. When comparing genetically modified mice, care was taken to alternate between conditions. Investigators were blinded when assessing adhesion scores in the peritoneal button adhesion model. Otherwise, no blinding was performed. Animals that died during experiments were excluded from analysis. If the abdominal cavity was accidentally opened during pouch intravital microscopy, animals were immediately euthanized and excluded from analysis. The number of times the experiment was replicated in the laboratory (N) as well as the number of biological replicates (n) is stated for each experiment in the figure legends. Generally, each data point represents one biological replicate if not explicitly state otherwise in the figure legend (FIG. 1M). All data are presented as mean (bar)+individual values. Statistical comparisons were performed using R statistical environment. Data were compared either by unpaired two-tailed t test or one-way or two-way ANOVA. Non-normally distributed variables were compared using unpaired two-samples Wilcoxon tests. Multiple comparisons were corrected with the Holm-Bonferroni method. A P-value of 0.05 was considered the threshold for significance.

### Example 2

Peritoneal Fluid Sample:

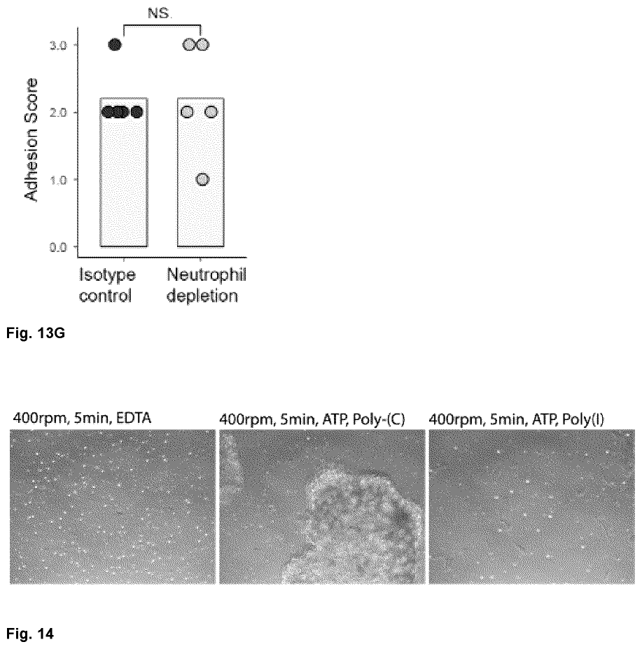

Cells were isolated from abdominal cavity by laparotomy or laparascopy, lavaged with 200 ml saline and stored on ice. The cell samples were transferred through 70 um filters into 50 ml falcon tubes and spun at 300 g for 5 min at 4 degree. The pellet was resuspended in HBSS without Ca and without Mg. For aggregometry cells were exposed to: EDTA final concentration 2 mM; ATPgs final concentration 100 uM; CaCl final concentration 2 mM; and Poly(I)/Poly(C) final concentration 2 mg/ml. The final volume of 100 uL was attained with HBSS. Cells from one patient were exposed to three different conditions: EDTA alone (negative control); ATP/Poly(C); ATP/Poly(I). Image with phase contrast was captured (FIG. 14).

Human Data:

When cells were exposed to Poly(I) there was no aggregates while in the Poly(C) there were large aggregate observed (FIG. 14). EDTA was used as a negative control.

### REFERENCES AND NOTES

1. H. Chisholm, ed. (1911), Coelom and Serous Membranes. Encyclopcedia Britannica 6 (11th ed.), 642-644 (1911).
2. K. Buchmann, Evolution of Innate Immunity: Clues from Invertebrates via Fish to Mammals. Front Immunol 5, 459 (2014).
3. K. A. Coffaro, R. T. Hinegardner, Immune response in the sea urchin Lytechinus pictus. Science (New York, N. Y) 197, 1389-1390 (1977).
4. Z. Pancer, Dynamic expression of multiple scavenger receptor cysteine-rich genes in coelomocytes of the purple sea urchin. Proceedings of the National Academy of Sciences of the United States of America 97, 13156-13161 (2000).
5. L. C. Smith, L. Chang, R. J. Britten, E. H. Davidson, Sea urchin genes expressed in activated coelomocytes are identified by expressed sequence tags. Complement homologues and other putative immune response genes suggest immune system homology within the deuterostomes. Journal of immunology (Baltimore, Md.: 1950) 156, 593-602 (1996).
6. Z. Pancer, J. P. Rast, E. H. Davidson, Origins of immunity: transcription factors and homologues of effector genes of the vertebrate immune system expressed in sea urchin coelomocytes. Immunogenetics 49, 773-786 (1999).
7. B. J. Hillier, V. D. Vacquier, Amassin, an olfactomedin protein, mediates the massive intercellular adhesion of sea urchin coelomocytes. The Journal of cell biology 160, 597-604 (2003).
8. P. T. Johnson, The coelomic elements of sea urchins (Strongylocentrotus). I. The normal coelomocytes; their morphology and dynamics in hanging drops. Journal of invertebrate pathology 13, 25-41 (1969).
9. E. E. Ghosn et al., Two physically, functionally, and developmentally distinct peritoneal macrophage subsets. Proceedings of the National Academy of Sciences of the United States of America 107, 2568-2573 (2010).
10. A. d. A. Cassado, M. R. D'Império Lima, K. R. Bortoluci, Revisiting Mouse Peritoneal Macrophages: Heterogeneity, Development, and Function. Frontiers in Immunology 6, 225 (2015).
11. N. Zhang et al., Expression of factor V by resident macrophages boosts host defense in the peritoneal cavity. The Journal of experimental medicine, jem.20182024 (2019).
12. J. Wang, P. Kubes, A Reservoir of Mature Cavity Macrophages that Can Rapidly Invade Visceral Organs to Affect Tissue Repair. Cell 165, 668-678 (2016).
13. S. Uderhardt, A. J. Martins, J. S. Tsang, T. Lämmermann, R. N. Germain, Resident Macrophages Cloak Tissue Microlesions to Prevent Neutrophil-Driven Inflammatory Damage. Cell 177, 541-555.e517 (2019).
14. J. F. Deniset et al., Gata6(+) Pericardial Cavity Macrophages Relocate to the Injured Heart and Prevent Cardiac Fibrosis. Immunity 51, 131-140.e135 (2019).
15. D. S. Nelson, Reaction to antigens in vivo of the peritoneal macrophages of guinea-pigs with delayed type hypersensitivity. Effects of anticoagulants and other drugs. Lancet 2, 175-176 (1963).
16. J. M. Weiler, R. E. Edens, R. J. Linhardt, D. P. Kapelanski, Heparin and modified heparin inhibit complement activation in vivo. The Journal of Immunology 148, 3210-3215 (1992).
17. J. L. Goldstein, Y. K. Ho, S. K. Basu, M. S. Brown, Binding site on macrophages that mediates uptake and degradation of acetylated low density lipoprotein, producing massive cholesterol deposition. Proceedings of the National Academy of Sciences of the United States of America 76, 333-337 (1979).
18. M. S. Brown, S. K. Basu, J. R. Falck, Y. K. Ho, J. L. Goldstein, The scavenger cell pathway for lipoprotein degradation: Specificity of the binding site that mediates the uptake of negatively-charged LDL by macrophages. Journal of Supramolecular Structure 13, 67-81 (1980).
19. B. Blumbach et al., The putative sponge aggregation receptor. Isolation and characterization of a molecule composed of scavenger receptor cysteine-rich domains and short consensus repeats. Journal of cell science 111 (Pt 17), 2635-2644 (1998).
20. D. M. Bowdish, S. Gordon, Conserved domains of the class A scavenger receptors: evolution and function. Immunological reviews 227, 19-31 (2009).
21. N. V. L. Yap, F. J. Whelan, D. M. E. Bowdish, G. B. Golding, The Evolution of the Scavenger Receptor Cysteine-Rich Domain of the Class A Scavenger Receptors. Frontiers in Immunology 6, (2015).
22. E. Sodergren et al., The Genome of the Sea Urchin <em> Strongylocentrotus purpuratus</em>. Science (New York, N. Y) 314, 941-952 (2006).
23. U. Brykczynska et al., Distinct Transcriptional Responses across Tissue-Resident Macrophages to Short-Term and Long-Term Metabolic Challenge. Cell reports 30, 1627-1643.e1627 (2020).
24. K. Coffaro, R. Hinegardner, Immune response in the sea urchin Lytechinus pictus. Science (New York, N.Y.) 197, 1389-1390 (1977).
25. F. S. Chia, J. Xing, Echinoderm coelomocytes. Zool. Stud. 35, 231-254 (1996).
26. B. W. Hellebrekers, T. Kooistra, Pathogenesis of postoperative adhesion formation. The British journal of surgery 98, 1503-1516 (2011).
27. V. Sikirica et al., The inpatient burden of abdominal and gynecological adhesiolysis in the US. BMC surgery 11, 13 (2011).
28. M. Nieuwenhuijzen, M. M. Reijnen, J. H. Kuijpers, H. van Goor, Small bowel obstruction after total or subtotal colectomy: a 10-year retrospective review. The British journal of surgery 85, 1242-1245 (1998).
29. W. Cates, T. M. Farley, P. J. Rowe, Worldwide patterns of infertility: is Africa different? Lancet 2, 596-598 (1985).
30. B. Kraemer et al., Standardised models for inducing experimental peritoneal adhesions in female rats. BioMed research international 2014, 435056 (2014).
31. J. M. Tsai et al., Surgical adhesions in mice are derived from mesothelial cells and can be targeted by antibodies against mesothelial markers. Science Translational Medicine 10, (2018).
32. S. R. Clark et al., Platelet TLR4 activates neutrophil extracellular traps to ensnare bacteria in septic blood. Nature medicine 13, 463-469 (2007).
33. C. H. Y. Wong, C. N. Jenne, B. Petri, N. L. Chrobok, P. Kubes, Nucleation of platelets with blood-borne pathogens on Kupffer cells precedes other innate immunity and contributes to bacterial clearance. Nature immunology 14, 785-792 (2013).
34. M. Stosik, W. DeptvBa, M. Trávnî ek, K. Baldy-Chudzik, Phagocytic and bactericidal activity of blood thrombocytes in carps (Cyprinus carpio). Veterinarni Medicina 47, 21-25 (2018).
35. T. Nagasawa et al., Phagocytosis by Thrombocytes is a Conserved Innate Immune Mechanism in Lower Vertebrates. Frontiers in Immunology 5, (2014).
36. M. B. Lawrence, T. A. Springer, Leukocytes roll on a selectin at physiologic flow rates: distinction from and prerequisite for adhesion through integrins. Cell 65, 859-873 (1991).
37. V. G. Martinez, S. K. Moestrup, U. Holmskov, J. Mollenhauer, F. Lozano, The conserved scavenger receptor cysteine-rich superfamily in therapy and diagnosis. Pharmacological reviews 63, 967-1000 (2011).
38. J. Zindel, P. Kubes, DAMPs, PAMPs, and LAMPs in Immunity and Sterile Inflammation. Annual review of pathology 15, 493-518 (2020).
39. L. Freyer et al., A loss-of-function and H2B-Venus transcriptional reporter allele for Gata6 in mice. BMC developmental biology 15, 38 (2015).
40. N. Faust, F. Varas, L. M. Kelly, S. Heck, T. Graf, Insertion of enhanced green fluorescent protein into the lysozyme gene creates mice with green fluorescent granulocytes and macrophages. Blood 96, 719-726 (2000).
41. A. Hasenberg et al., Catchup: a mouse model for imaging-based tracking and modulation of neutrophil granulocytes. Nature methods 12, 445-452 (2015).
42. J. Zhang et al., CD41-YFP mice allow in vivo labeling of megakaryocytic cells and reveal a subset of platelets hyperreactive to thrombin stimulation. Experimental hematology 35, 490-499 (2007).
43. K. Y. Helmy et al., CRIg: a macrophage complement receptor required for phagocytosis of circulating pathogens. Cell 124, 915-927 (2006).
44. H. A. Schreiber et al., Intestinal monocytes and macrophages are required for T cell polarization in response to Citrobacter rodentium. The Journal of experimental medicine 210, 2025-2039 (2013).
45. B. G. J. Surewaard, P. Kubes, Measurement of bacterial capture and phagosome maturation of Kupffer cells by intravital microscopy. Methods (San Diego, Calf) 128, 12-19 (2017).
46. B. McDonald et al., Intravascular Danger Signals Guide Neutrophils to Sites of Sterile Inflammation. Science (New York, N. Y) 330, 362-366 (2010).
47. E. Bianchi et al., Ghrelin ameliorates adhesions in a postsurgical mouse model. The Journal of surgical research 201, 226-234 (2016).
48. L. Ritsma et al., Surgical implantation of an abdominal imaging window for intravital microscopy. Nature protocols 8, 583-594 (2013).
49. D. Dal-Secco et al., A dynamic spectrum of monocytes arising from the in situ reprogramming of CCR2+ monocytes at a site of sterile injury. The Journal of experimental medicine 212, 447-456 (2015).
50. K. D. Nolph, Z. J. Twardowski, R. P. Popovich, J. Rubin, Equilibration of peritoneal dialysis solutions during long-dwell exchanges. The Journal of laboratory and clinical medicine 93, 246-256 (1979).
51. H. V. Zuhlke, E. M. Lorenz, E. M. Straub, V. Savvas, [Pathophysiology and classification of adhesions]. Langenbecks Archiv fur Chirurgie. Supplement II, Verhandlungen der Deutschen Gesellschaft fur Chirurgie. Deutsche Gesellschaft fur Chirurgie. Kongress, 1009-1016 (1990).
52. A. Klingberg et al., Fully Automated Evaluation of Total Glomerular Number and Capillary Tuft Size in Nephritic Kidneys Using Lightsheet Microscopy. Journal of the American Society of Nephrology: JASN 28, 452-459 (2017).
53. C. Deppermann et al., Gray platelet syndrome and defective thrombo-inflammation in Nbeal2-deficient mice. The Journal of clinical investigation 123, 3331-3342 (2013).
54. C. W. Law et al., RNA-seq analysis is easy as 1-2-3 with limma, Glimma and edgeR. F1000Research 5, ISCB Comm J-1408 (2016).
55. J. Schindelin et al., Fiji: an open-source platform for biological-image analysis. Nature methods 9, 676-682 (2012).
56. J. Y. Tinevez et al., TrackMate: An open and extensible platform for single-particle tracking. Methods (San Diego, Calf) 115, 80-90 (2017).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of treating a subject having or suspected of having a disease or disorder associated with peritoneal adhesions, comprising: administering an inhibitor of a scavenger receptor comprising a SRCR domain recognizing negatively charged macromolecules.

2. A method of treating a subject having or suspected of having a disease or disorder associated with pericardium adhesions, comprising: administering an inhibitor of a scavenger receptor comprising a SRCR domain recognizing negatively charged macromolecules.

3. A method of treating a subject having or suspected of having a disease or disorder associated with pleural cavity adhesions, comprising: administering an inhibitor of a scavenger receptor comprising a SRCR domain recognizing negatively charged macromolecules.

4. A method of treating a subject having or suspected of having peritoneal adhesions, comprising: administering an inhibitor of a scavenger receptor comprising a SRCR domain.

5. A method of treating a subject having or suspected of having pericardium adhesions, comprising: administering an inhibitor of a scavenger receptor comprising a SRCR domain.

6. A method of treating a subject having or suspected of having plural cavity adhesions, comprising: administering an inhibitor of a scavenger receptor comprising a SRCR domain.

7. The method of claim 1, wherein the disease or disorder associated with peritoneal adhesions comprises adhesions that results from abdomino-pelvic surgery, radiation therapy, and/or inflammatory processes.

8. The method of claim 2, wherein the disease or disorder associated with pericardium adhesions comprises adhesions that results from abdomino-pelvic surgery, radiation therapy, and/or inflammatory processes.

9. The method of claim 3, wherein the disease or disorder associated with pleural cavity adhesions comprises adhesions that results from abdomino-pelvic surgery, radiation therapy, and/or inflammatory processes.

10. The method of any one of claims 1, 4, or 7, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

11. The method of any one of claims 1 to 10, wherein said inhibitor comprises or consists of Polyl.

12. The method of any one of claims 1 to 10, where said inhibitor comprises or consists of an antibody.

13. The method of claim 12, wherein said antibody is a polyclonal antibody or a monoclonal antibody.

14. The method of any one of claims 1 to 13, wherein the subject is a human.

15. Use of an inhibitor of a scavenger receptor comprising a SRCR domain recognizing negatively charged macromolecules, for treating a subject having or suspected of having a disease or disorder associated with peritoneal adhesions.

16. Use of an inhibitor of a scavenger receptor comprising a SRCR domain recognizing negatively charged macromolecules, in the manufacture of a medicament for treating a subject having or suspected of having a disease or disorder associated with peritoneal adhesions.

17. Use of an inhibitor of a scavenger receptor comprising a SRCR domain recognizing negatively charged macromolecules for treating a subject having or suspected of having a disease or disorder associated with pericardium adhesions.

18. Use of an inhibitor of a scavenger receptor comprising a SRCR domain recognizing negatively charged macromolecules in the manufacture of a medicament for treating a subject having or suspected of having a disease or disorder associated with pericardium adhesions.

19. Use of an inhibitor of a scavenger receptor comprising a SRCR domain recognizing negatively charged macromolecules treating a subject having or suspected of having a disease or disorder associated with pleural cavity adhesions.

20. Use of an inhibitor of a scavenger receptor comprising a SRCR domain recognizing negatively charged macromolecules in the manufacture of a medicament for treating a subject having or suspected of having a disease or disorder associated with pleural cavity adhesions.

21. Use of an inhibitor of a scavenger receptor comprising a SRCR domain for treating a subject having or suspected of having peritoneal adhesions.

22. Use of an inhibitor of a scavenger receptor comprising a SRCR domain in the manufacture of a medicament for treating a subject having or suspected of having peritoneal adhesions.

23. Use of an inhibitor of a scavenger receptor comprising a SRCR domain for treating a subject having or suspected of having pericardium adhesions.

24. Use of an inhibitor of a scavenger receptor comprising a SRCR domain in the manufacture of a medicament for treating a subject having or suspected of having pericardium adhesions.

25. Use of a scavenger receptor comprising a SRCR domain for treating a subject having or suspected of having plural cavity adhesions.

26. The use of claim 15 or 16, wherein the disease or disorder associated with peritoneal adhesions comprises adhesions that results from abdomino-pelvic surgery, radiation therapy, and/or inflammatory processes.

27. The use of claim 17 or 18, wherein the disease or disorder associated with pericardium adhesions comprises adhesions that results from abdomino-pelvic surgery, radiation therapy, and/or inflammatory processes.

28. The use of claim 19 or 20, wherein the disease or disorder associated with pleural cavity adhesions comprises adhesions that results from abdomino-pelvic surgery, radiation therapy, and/or inflammatory processes.

29. The use of any one of claims 15, 16, 21, 22, 26, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

30. The use of any one of claims 15 to 29, wherein said inhibitor comprises or consists of Polyl.

31. The use of any one of claims 15 to 29, where said inhibitor comprises or consists of an antibody.

32. The method of claim 31, wherein said antibody is a polyclonal antibody or a monoclonal antibody.

33. The method of any one of claims 15 to 32, wherein the subject is a human.

34. A kit for treating a subject having or suspected of having a disease or disorder associated with peritoneal adhesions, comprising: an inhibitor of a scavenger receptor comprising a SRCR domain recognizing negatively charged macromolecules, a container, and optionally instructions for the use thereof.

35. A kit for treating a subject having or suspected of having a disease or disorder associated with pericardium adhesions, comprising: an inhibitor of a scavenger receptor comprising a SRCR domain recognizing negatively charged macromolecules, a container, and optionally instructions for the use thereof.

36. A kit for treating a subject having or suspected of having a disease or disorder associated with pleural cavity adhesions, comprising: an inhibitor of a scavenger receptor comprising a SRCR domain recognizing negatively charged macromolecules, a container, and optionally instructions for the use thereof.

37. A kit for treating a subject having or suspected of having peritoneal adhesions, comprising: an inhibitor of a scavenger receptor comprising a SRCR domain, a container, and optionally instructions for the use thereof.

38. A kit for treating a subject having or suspected of having pericardium adhesions, comprising: an inhibitor of a scavenger receptor comprising a SRCR domain, a container, and optionally instructions for the use thereof.

39. A kit for treating a subject having or suspected of having plural cavity adhesions, comprising: an inhibitor of a scavenger receptor comprising a SRCR domain, a container, and optionally instructions for the use thereof.

40. The kit of claim 34, wherein the disease or disorder associated with peritoneal adhesions comprises adhesions that results from abdomino-pelvic surgery, radiation therapy, and/or inflammatory processes.

41. The kit of claim 35, wherein the disease or disorder associated with pericardium adhesions comprises adhesions that results from abdomino-pelvic surgery, radiation therapy, and/or inflammatory processes.

42. The kit of claim 36, wherein the disease or disorder associated with pleural cavity adhesions comprises adhesions that results from abdomino-pelvic surgery, radiation therapy, and/or inflammatory processes.

43. The kit of any one of claims 34, 37, or 40, wherein treatment reduces or prevents a symptom associated with said disease or disorder associated with peritoneal adhesions selected from chronic (persistent or intermittent) bloating, abdominal cramping or borborygmi, altered bowel habits including constipation, frequent stools, nausea with our without early satiety, bowel obstructions which may be transient, partial, or complete, female infertility and dyspareunia, rectal bleeding or dyschezia during menses.

44. The kit of any one of claims 34 to 43, wherein said inhibitor comprises or consists of Polyl.

45. The kit of any one of claims 34 to 43, where said inhibitor comprises or consists of an antibody.

46. The method of claim 45, wherein said antibody is a polyclonal antibody or a monoclonal antibody.

47. The method of any one of claims 34 to 46, wherein the subject is a human.
